SEQUENCE LISTING (55 sequences)

SEQ ID NO: 1 - protein, 130 aa, Saccharomyces cerevisiae
MSQPVQRAAARSFLQKYINKETLKYIFTTHFWGPVSNFGIPIAAIYDLKKDPTLISGPMTFALVTYSGVFMKYALSVSPKNYLLFGCHLINETAQLAQGYRFLKYTYFTTDEEKKALDKEWKEKEKTGKQ

SEQ ID NO: 2 - protein, 129 aa, Saccharomyces cerevisiae
MSTSSVRFAFRRFWQSETGPKTVHFWAPTLKWGLVFAGFSDMKRPVEKISGAQNLSLLSTALIWTRWSFVIKPRNILLASVNSFLCLTAGYQLGRIANYRIRNGDSISQLCSYILSGADESKKEITTGR

SEQ ID NO: 3 - protein, 147 aa, Saccharomyces cerevisiae
MSASAFNFAFRRFWNSETGPKTVHFWAPTLKWGLVFAGLNDIKRPVEKVSGAQNLSLLATALIWTRWSFVIKPKNYLLASVNFFLGCTAGYHLTRIANFRIRNGDSFKQVIHYIIKGETPAAVAAKQTASTSMNKGVIGTNPPITHR

SEQ ID NO: 4 - DNA, 393 bp, Saccharomyces cerevisiae
atgtctcaaccggttcaacgcgctgcagcacgctcattccttcaaaaatacatcaataaagaaactttgaaatatattttcacaacacacttctggggtcccgtatcaaatttcggtatcccaattgctgctatatatgatctgaaaaaagaccctacactaatctctggcccaatgacttttgctttagttacctattcaggtgttttcatgaagtatgctctttcagtatcacccaaaaactacttactgtttggatgccaccttattaatgaaactgcgcaattagctcaaggctataggtttctcaaatacacgtatttcacaacagatgaggagaagaaagctctagataaggaatggaaagagaaagaaaaaactggtaaacagtaa

SEQ ID NO: 5 - DNA, 390 bp, Saccharomyces cerevisiae
atgtctacatcatccgtacgttttgcatttaggcggttctggcaaagtgagacaggccccaagacggtgcatttctgggctcctactttgaaatggggtctggttttcgctggattcagcgatatgaagagaccggtggaaaaaatttctggtgctcaaaatttgtcgctgctatctactgcgctgatttggactcgttggtcctttgtcatcaagccaagaaacatcttgttggcttctgtcaactcgtttctttgtctgaccgctggctatcaattgggtagaattgccaactacaggatacggaatggcgactctatatcgcaattgtgtagctatattctcagcggcgccgacgaaagcaaaaaggaaattactacgggcagataa

SEQ ID NO: 6 - DNA, 441 bp, Saccharomyces cerevisiae
atgtcagcatcagcttttaattttgcctttagaagattttggaatagtgaaacaggccctaaaacagtacacttctgggccccaactttgaagtgggggctggtcttcgcagggctaaatgatattaagaggcctgttgagaaggtatcaggagcacaaaatttatctttattagcgacggcactgatttggacgcgttggtcgtttgtcatcaagcccaagaactatctgttagcttccgtcaattttttcctgggttgcactgcaggctaccatctaacaagaattgctaactttaggatacggaacggtgattcttttaaacaggttattcactacataataaaaggggagactcctgcagccgtcgcagcaaagcaaactgcatccacatcgatgaacaaaggtgtgatcggtactaatccgccaataacgcactga

SEQ ID NO: 7 - protein, 332 aa, Pelodiscus sinensis japonicus
MSVKELLIQNVHKEEHSHAHNKITVVGVGAVGMACAISILMKDLADELALVDVIEDKLRGEMLDLQHGSLFLRTPKIVSGKDYSVTAHSKLVIITAGARQQEGESRLNLVQRNVNIFKFIIPNVVKYSPDCMLLVVSNPVDILTYVAWKISGFPKHRVIGSGCNLDSARFRYLMGEKLGIHSLSCHGWIIGEHGDSSVPVWSGVNVAGVSLKALYPDLGTDADKEHWKEVHKQVVDSAYEVIKLKGYTSWAIGLSVADLAETVMKNLRRVHPISTMVKGMYGVSSDVFLSVPCVLGYAGITDVVKMTLKSEEEEKLRKSADTLWGIQKELQF

SEQ ID NO: 8 - protein, 332 aa, Ornithorhynchus anatinus
MAGVKEQLIQNLLKEEYAPQNKITVVGVGAVGMACAISILMKDLADELALVDVIEDKLKGEMMDLQHGSLFLRTPKIVSGKDYSVTANSKLVIITAGARQQEGESRLNLVQRNVNIFKFIIPNVVKYSPNCKLLVVSNPVDILTYVAWKISGFPKNRVIGSGCNLDSARFRYLMGERLGIHSTSCHGWVIGEHGDSSVPVWSGVNVAGVSLKNLHPDLGTDADKEQWKDVHKQVVDSAYEVIKLKGYTSWAIGLSVADLAESIVKNLRRVHPISTMIKGLYGIKDEVFLSVPCVLGQNGISDVVKITLKSEEEAHLKKSADTLWGIQKELQF

SEQ ID NO: 9 - protein, 332 aa, Tursiops truncatus
MATVKDQLIQNLLKEEHVPQNKITVVGVGAVGMACAISILMKDLADELALVDVIEDKLKGEMMDLQHGSLFLRTPKIVSGKDYSVTANSKLVIITAGARQQEGESRLNLVQRNVNIFKFIVPNIVKYSPHCKLLVVSNPVDILTYVAWKISGFPKNRVIGSGCNLDSARFRYLMGERLGVHPLSCHGWILGEHGDSSVPVWSGVNVAGVSLKNLHPELGTDADKEHWKAIHKQVVDSAYEVIKLKGYTSWAVGLSVADLAESIMKNLRRVHPISTMIKGLYGIKEDVFLSVPCILGQNGISDVVKVTLTPEEQACLKKSADTLWGIQKELQF

SEQ ID NO: 10 - protein, 332 aa, Rattus norvegicus
MAALKDQLIVNLLKEEQVPQNKITVVGVGAVGMACAISILMKDLADELALVDVIEDKLKGEMMDLQHGSLFLKTPKIVSSKDYSVTANSKLVIITAGARQQEGESRLNLVQRNVNIFKFIIPNVVKYSPQCKLLIVSNPVDILTYVAWKISGFPKNRVIGSGCNLDSARFRYLMGERLGVHPLSCHGWVLGEHGDSSVPVWSGVNVAGVSLKSLNPQLGTDADKEQWKDVHKQVVDSAYEVIKLKGYTSWAIGLSVADLAESIMKNLRRVHPISTMIKGLYGIKEDVFLSVPCILGQNGISDVVKVTLTPDEEARLKKSADTLWGIQKELQF

SEQ ID NO: 11 - DNA, 999 bp, Pelodiscus sinensis japonicus
atgtccgtaaaggaactacttatacaaaacgtccataaggaggagcattctcacgctcacaataagataacagttgtaggagtaggtgcagtaggtatggcatgtgctatttcgatattaatgaaagacttggctgatgaactagccttggttgatgtgattgaggataagttacgtggagaaatgttagatttgcaacatggttcattgttcttgagaacccccaaaattgtctcgggtaaggattattcagtcactgctcattctaaactggttatcattacagcaggtgcaagacagcaagaaggggagagcagactaaatctggttcaacgtaatgtcaacatcttcaagtttatcatcccgaacgtagtaaaatacagtccagactgcatgttgcttgttgtgagtaatccagttgacatcttaacctatgttgcgtggaaaatcagtgggtttccaaaacatagggtgattggctcaggatgcaaccttgatagcgccaggtttaggtatctaatgggagaaaaattaggtattcactccttatcttgtcatggctggataataggcgaacatggtgattcttcggtacctgtttggtccggggttaatgtggctggtgttagtttaaaagcattatatcctgacctgggtactgatgccgataaagaacattggaaagaagtgcacaaacaagtggttgattctgcttacgaagttattaaacttaagggctacacttcttgggctataggtctatcagtagctgatttggcagaaaccgttatgaaaaatttaagaagagtccacccaatttccacgatggtcaagggtatgtacggtgttagctctgacgtcttcttatctgttccttgtgttttgggatatgcgggaattacagacgtcgtgaagatgacattgaaatcagaggaagaggaaaaactaagaaagtcagccgatactctgtggggcattcaaaaggaattgcagttttaa

SEQ ID NO: 12 - DNA, 1267 bp, Artificial Sequence
  Synthetic (ARS/CEN)
gagctcctttcatttctgataaaagtaagattactccatttatcttttcaccaacatattcatagttgaaagttatccttctaagtacgtatacaatattaattaaacgtaaaaacaaaactgactgtaaaaatgtgtaaaaaaaaaatatcaaattcatagcagtttcaaggaatgaaaactattatgatctggtcacgtgtatataaattattaattttaaacccatataatttattatttttttattctaaagtttaaagtaattttagtagtattttatattttgaataaatatactttaaatttttatttttatattttattacttttaaaaataatgtttttatttaaaacaaaattataagttaaaaagttgttccgaaagtaaaatatattttatagtttttacaaaaataaattatttttaacgtattttttttaattatatttttgtatgtgattatatccacaggtattatgctgaatttagctgtttcagtttaccagtgtgatagtatgattttttttgcctctcaaaagctatttttttagaagcttcgtcttagaaataggtggtgtataaattgcggttgacttttaactatatatcattttcgatttatttattacatagagaggtgcttttaattttttaatttttattttcaataattttaaaagtgggtacttttaaattggaacaaagtgaaaaatatctgttatacgtgcaactgaattttactgaccttaaaggactatctcaatcctggttcagaaatccttgaaatgattgatatgttggtggattttctctgattttcaaacaagaggtattttatttcatatttattatattttttacatttattttatatttttttattgtttggaagggaaagcgacaatcaaattcaaaatatattaattaaactgtaatacttaataagagacaaataacagccaagaatcaaatactgggtttttaatcaaaagatctctctacatgcacccaaattcattatttaaatttactatactacagacagaatatacgaacccagattaagtagtcagacgcttttccgctttattgagtatatagccttacatattttctgcccataatttctggatttaaaataaacaaaaatggttactttgtagttatgaaaaaaggcttttccaaaatgcgaaatacgtgttatttaaggttaatcaacaaaacgcatatccatatgggtagttggacaaaacttcaatcgat

SEQ ID NO: 13 - DNA, 289 bp, Artificial Sequence
  Synthetic (CYC promoter)
atttggcgagcgttggttggtggatcaagcccacgcgtaggcaatcctcgagcagatccgccaggcgtgtatatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacgcaaacacaaatacacacactaa

SEQ ID NO: 14 - DNA, 401 bp, Artificial Sequence
  Synthetic (TEF promoter)
atagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttt

SEQ ID NO: 15 - DNA, 655 bp, Artificial Sequence
  Synthetic (GPD promoter)
agtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggat

SEQ ID NO: 16 - DNA, 1468 bp, Artificial Sequence
  Synthetic (ADH promoter)
gccgggatcgaagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactccaagctggccgc

SEQ ID NO: 17 - DNA, 252 bp, Artificial Sequence
  Synthetic (CYC1 terminator)
tcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggcc

SEQ ID NO: 18 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 19 - DNA, 1692 bp, Saccharomyces cerevisiae
atgtctgaaattactttgggtaaatatttgttcgaaagattaaagcaagtcaacgttaacaccgttttcggtttgccaggtgacttcaacttgtccttgttggacaagatctacgaagttgaaggtatgagatgggctggtaacgccaacgaattgaacgctgcttacgccgctgatggttacgctcgtatcaagggtatgtcttgtatcatcaccaccttcggtgtcggtgaattgtctgctttgaacggtattgccggttcttacgctgaacacgtcggtgttttgcacgttgttggtgtcccatccatctctgctcaagctaagcaattgttgttgcaccacaccttgggtaacggtgacttcactgttttccacagaatgtctgccaacatttctgaaaccactgctatgatcactgacattgctaccgccccagctgaaattgacagatgtatcagaaccacttacgtcacccaaagaccagtctacttaggtttgccagctaacttggtcgacttgaacgtcccagctaagttgttgcaaactccaattgacatgtctttgaagccaaacgatgctgaatccgaaaaggaagtcattgacaccatcttggctttggtcaaggatgctaagaacccagttatcttggctgatgcttgttgttccagacacgacgtcaaggctgaaactaagaagttgattgacttgactcaattcccagctttcgtcaccccaatgggtaagggttccattgacgaacaacacccaagatacggtggtgtttacgtcggtaccttgtccaagccagaagttaaggaagccgttgaatctgctgacttgattttgtctgtcggtgctttgttgtctgatttcaacaccggttctttctcttactcttacaagaccaagaacattgtcgaattccactccgaccacatgaagatcagaaacgccactttcccaggtgtccaaatgaaattcgttttgcaaaagttgttgaccactattgctgacgccgctaagggttacaagccagttgctgtcccagctagaactccagctaacgctgctgtcccagcttctaccccattgaagcaagaatggatgtggaaccaattgggtaacttcttgcaagaaggtgatgttgtcattgctgaaaccggtacctccgctttcggtatcaaccaaaccactttcccaaacaacacctacggtatctctcaagtcttatggggttccattggtttcaccactggtgctaccttgggtgctgctttcgctgctgaagaaattgatccaaagaagagagttatcttattcattggtgacggttctttgcaattgactgttcaagaaatctccaccatgatcagatggggcttgaagccatacttgttcgtcttgaacaacgatggttacaccattgaaaagttgattcacggtccaaaggctcaatacaacgaaattcaaggttgggaccacctatccttgttgccaactttcggtgctaaggactatgaaacccacagagtcgctaccaccggtgaatgggacaagttgacccaagacaagtctttcaacgacaactctaagatcagaatgattgaaatcatgttgccagtcttcgatgctccacaaaacttggttgaacaagctaagttgactgctgctaccaacgctaagcaataa

SEQ ID NO: 20 - protein, 591 aa, Saccharomyces cerevisiae
MLKYKPLLKISKNCEAAILRASKTRLNTIRAYGSTVPKSKSFEQDSRKRTQSWTALRVGAILAATSSVAYLNWHNGQIDNEPKLDMNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPKILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEKAIEILRDEIEMSMRLLGVTSIAELKPDLLDLSTLKARTVGVPNDVLYNEVYEGPTLTEFEDA

SEQ ID NO: 21 - DNA, 1776 bp, Saccharomyces cerevisiae
atgctaaaatacaaacctttactaaaaatctcgaagaactgtgaggctgctatcctcagagcgtctaagactagattgaacacaatccgcgcgtacggttctaccgttccaaaatccaagtcgttcgaacaagactcaagaaaacgcacacagtcatggactgccttgagagtcggtgcaattctagccgctactagttccgtggcgtatctaaactggcataatggccaaatagacaacgagccgaaactggatatgaataaacaaaagatttcgcccgctgaagttgccaagcataacaagcccgatgattgttgggttgtgatcaatggttacgtatacgacttaacgcgattcctaccaaatcatccaggtgggcaggatgttatcaagtttaacgccgggaaagatgtcactgctatttttgaaccactacatgctcctaatgtcatcgataagtatatagctcccgagaaaaaattgggtccccttcaaggatccatgcctcctgaacttgtctgtcctccttatgctcctggtgaaactaaggaagatatcgctagaaaagaacaactaaaatcgctgctacctcctctagataatattattaacctttacgactttgaatacttggcctctcaaactttgactaaacaagcgtgggcctactattcctccggtgctaacgacgaagttactcacagagaaaaccataatgcttatcataggatttttttcaaaccaaagatccttgtagatgtacgcaaagtagacatttcaactgacatgttgggttctcatgtggatgttcccttctacgtgtctgctacagctttgtgtaaactgggaaaccccttagaaggtgaaaaagatgtcgccagaggttgtggccaaggtgtgacaaaagtcccacaaatgatatctactttggcttcatgttcccctgaggaaattattgaagcagcaccctctgataaacaaattcaatggtaccaactatatgttaactctgatagaaagatcactgatgatttggttaaaaatgtagaaaagctgggtgtaaaggcattatttgtcactgtggatgctccaagtttaggtcaaagagaaaaagatatgaagctgaaattttccaatacaaaggctggtccaaaagcgatgaagaaaactaatgtagaagaatctcaaggtgcttcgagagcgttatcaaagtttattgacccctctttgacttggaaagatatagaagagttgaagaaaaagacaaaactacctattgttatcaaaggtgttcaacgtaccgaagatgttatcaaagcagcagaaatcggtgtaagtggggtggttctatccaatcatggtggtagacaattagatttttcaagggctcccattgaagtcctggctgaaaccatgccaatcctggaacaacgtaacttgaaggataagttggaagttttcgtggacggtggtgttcgtcgtggtacagatgtcttgaaagcgttatgtctaggtgctaaaggtgttggtttgggtagaccattcttgtatgcgaactcatgctatggtcgtaatggtgttgaaaaagccattgaaattttaagagatgaaattgaaatgtctatgagactattaggtgttactagcattgcggaattgaagcctgatcttttagatctatcaacactaaaggcaagaacagttggagtaccaaacgacgtgctgtataatgaagtttatgagggacctactttaacagaatttgaggatgcatga

SEQ ID NO: 22 - protein, 391 aa, Saccharomyces cerevisiae
MSAAADRLNLTSGHLNAGRKRSSSSVSLKAAEKPFKVTVIGSGNWGTTIAKVVAENCKGYPEVFAPIVQMWVFEEEINGEKLTEIINTRHQNVKYLPGITLPDNLVANPDLIDSVKDVDIIVFNIPHQFLPRICSQLKGHVDSHVRAISCLKGFEVGAKGVQLLSSYITEELGIQCGALSGANIATEVAQEHWSETTVAYHIPKDFRGEGKDVDHKVLKALFHRPYFHVSVIEDVAGISICGALKNVVALGCGFVEGLGWGNNASAAIQRVGLGEIIRFGQMFFPESREETYYQESAGVADLITTCAGGRNVKVARLMATSGKDAWECEKELLNGQSAQGLITCKEVHEWLETCGSVEDFPLFEAVYQIVYNNYPMKNLPDMIEELDLHED

SEQ ID NO: 23 - DNA, 1176 bp, Saccharomyces cerevisiae
atgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattag

SEQ ID NO: 24 - protein, 347 aa, Saccharomyces cerevisiae
MSIPETQKGVIFYESHGKLEYKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIGSYVGNRADTREALDFFARGLVKSPIKVVGLSTLPEIYEKMEKGQIVGRYVVDTSK

SEQ ID NO: 25 - DNA, 1047 bp, Saccharomyces cerevisiae
atgtctatcccagaaactcaaaaaggtgttatcttctacgaatcccacggtaagttggaatacaaagatattccagttccaaagccaaaggccaacgaattgttgatcaacgttaaatactctggtgtctgtcacactgacttgcacgcttggcacggtgactggccattgccagttaagctaccattagtcggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaacaatacgctaccgctgacgctgttcaagccgctcacattcctcaaggtaccgacttggcccaagtcgcccccatcttgtgtgctggtatcaccgtctacaaggctttgaagtctgctaacttgatggccggtcactgggttgctatctccggtgctgctggtggtctaggttctttggctgttcaatacgccaaggctatgggttacagagtcttgggtattgacggtggtgaaggtaaggaagaattattcagatccatcggtggtgaagtcttcattgacttcactaaggaaaaggacattgtcggtgctgttctaaaggccactgacggtggtgctcacggtgtcatcaacgtttccgtttccgaagccgctattgaagcttctaccagatacgttagagctaacggtaccaccgttttggtcggtatgccagctggtgccaagtgttgttctgatgtcttcaaccaagtcgtcaagtccatctctattgttggttcttacgtcggtaacagagctgacaccagagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataa

SEQ ID NO: 26 - protein, 560 aa, Saccharomyces cerevisiae
MIRQSLMKTVWANSSRFSLQSKSGLVKYAKNRSFHAARNLLEDKKVILQKVAPTTGVVAKQSFFKRTGKFTLKALLYSALAGTAYVSYSLYREANPSTQVPQSDTFPNGSKRKTLVILGSGWGSVSLLKNLDTTLYNVVVVSPRNYFLFTPLLPSTPVGTIELKSIVEPVRTIARRSHGEVHYYEAEAYDVDPENKTIKVKSSAKNNDYDLDLKYDYLVVGVGAQPNTFGTPGVYEYSSFLKEISDAQEIRLKIMSSIEKAASLSPKDPERARLLSFVVVGGGPTGVEFAAELRDYVDQDLRKWMPELSKEIKVTLVEALPNILNMFDKYLVDYAQDLFKEEKIDLRLKTMVKKVDATTITAKTGDGDIENIPYGVLVWATGNAPREVSKNLMTKLEEQDSRRGLLIDNKLQLLGAKGSIFAIGDCTFHPGLFPTAQVAHQEGEYLAQYFKKAYKIDQLNWKMTHAKDDSEVARLKNQIVKTQSQIEDFKYNHKGALAYIGSDKAIADLAVGEAKYRLAGSFTFLFWKSAYLAMCLSFRNRVLVAMDWAKVYFLGRDSSI

SEQ ID NO: 27 - protein, 545 aa, Saccharomyces cerevisiae
MLPRLGFARTARSIHRFKMTQISKPFFHSTEVGKPGPQQKLSKSYTAVFKKWFVRGLKLTFYTTLAGTLYVSYELYKESNPPKQVPQSTAFANGLKKKELVILGTGWGAISLLKKLDTSLYNVTVVSPRSFFLFTPLLPSTPVGTIEMKSIVEPVRSIARRTPGEVHYIEAEALDVDPKAKKVMVQSVSEDEYFVSSLSYDYLVVSVGAKTTTFNIPGVYGNANFLKEIEDAQNIRMKLMKTIEQASSFPVNDPERKRLLTFVVVGGGPTGVEFAAELQDYINQDLRKWMPDLSKEMKVILIEALPNILNMFDKTLIKYAEDLFARDEIDLQVNTAVKVVEPTYIRTLQNGQTNTDIEYGMLVWATGNEPIDFSKTLMSRIPEQTNRRGLLINDKLELLGSENSIYAIGDCTAHTGFFPTAQVAHQEGEYLAKILDKKLQIEQLEWDMLNSTDETEVSRLQKEVNLRKSKLDKFNYKHMGALAYIGSETAIADLHMGDSSYQLKGMFAFLFWKSAYLAMCLSIRNRILIAMDWTKVYFLGRDSSV

SEQ ID NO: 28 - DNA, 1683 bp, Saccharomyces cerevisiae
atgattagacaatcattaatgaaaacagtgtgggctaactcctccaggtttagcctacagagcaagtcggggcttgtgaaatatgccaaaaatagatcgttccatgcagcaagaaatttgctagaggacaagaaagtcattttgcaaaaagtggcgcccactactggcgttgttgcgaagcagtcctttttcaagagaactgggaaatttactttgaaggctttattgtattctgccctcgcgggtacggcttacgtttcatactcactttaccgagaagctaacccttctacccaagttcctcaatcggacacttttccaaacggttcaaagaggaagactttggtaattctgggctccggttggggttctgtgtcgcttttgaaaaatttggacaccacgttgtataatgttgttgttgtttctccaagaaattattttctttttactccgctattgccatctaccccagttggtaccatcgaattgaaatctattgttgaacctgtcaggactattgctagaagatcgcacggtgaagtccattactatgaagctgaagcgtacgacgttgatcctgaaaacaaaacaattaaggtcaaatcttccgctaagaataacgactacgacttggacttgaaatacgactatctggttgtcggtgtgggtgctcaaccaaacacttttggtactccgggagtttatgaatattcttctttcttgaaggaaatatccgacgctcaagagatcagattaaaaattatgtccagtattgagaaagctgcctccctatctccaaaagatcctgagagagcaagattgttgagctttgttgtcgttggtggtggtcccaccggtgtcgaatttgccgctgaattgagagattatgttgaccaggacttgagaaaatggatgcccgaattgagtaaagaaattaaagtcactttggtggaggctttgccaaacattttgaacatgtttgacaagtatctcgttgactatgctcaagatttattcaaagaggaaaaaatcgatttaagattgaaaacaatggttaagaaagttgacgctaccactataactgccaaaactggcgatggtgacattgaaaatataccgtatggtgtattagtttgggctacaggtaatgcgccaagagaagtgtctaagaacctaatgactaaattagaggaacaggactcaagacgtggtttgttgatagataacaaacttcaacttttgggtgctaagggatctatttttgctatcggcgattgtaccttccaccctggcttgttccctaccgctcaagttgcccaccaagaaggtgaatacttggctcagtatttcaagaaagcttataaaatcgatcaattgaactggaaaatgacccatgctaaagacgattcagaagtcgctagattaaagaaccaaatagtcaaaacgcaatcgcaaattgaagacttcaagtacaaccataagggtgctctggcttatattggttcagataaagccattgctgatcttgccgttggtgaagccaaatataggttagccggctcattcaccttcctattctggaaatctgcttatttggcaatgtgtctatcctttagaaacagagttcttgtcgctatggattgggctaaagtttatttcttgggtagagattcatctatctag

SEQ ID NO: 29 - DNA, 1638 bp, Saccharomyces cerevisiae
atgctgcccagacttggttttgcgaggactgctaggtccatacaccgtttcaagatgacccagatctctaaaccttttttccattccactgaagttggtaagcccggaccacagcagaagctatcgaaatcttacactgcggtattcaagaaatggtttgtcagaggtttaaagttaaccttttacacgacgttggccggcacattgtatgtgtcatacgagctgtacaaagaatcgaacccacccaaacaggttccccaatcgaccgcttttgctaatggtttgaaaaagaaggagctggttattttgggtacaggctggggcgccatatctcttttgaagaaattagacacgtctttgtataacgtgaccgtggtgtcgccaagaagcttctttttgttcacaccgttattaccctcaacgcctgtgggtacgatagagatgaagtctattgtcgaaccggttagatcgatcgctagaagaacgcctggagaagttcactacattgaggcggaagcgttggacgttgatccaaaggccaaaaaagtaatggtgcaatcggtgtcagaggacgaatatttcgtttcgagcttaagttacgattatcttgttgttagtgtaggcgctaaaaccactacttttaacattcccggggtctatggcaatgctaacttcttgaaagagattgaagatgctcaaaatattcgtatgaagttaatgaaaaccatagaacaggcaagttcatttcctgtgaacgatccggaaaggaagcgattattaacgttcgtggttgttggagggggccctacgggggttgaatttgccgccgaactgcaagattacatcaatcaagatttgaggaagtggatgcccgacttaagtaaagaaatgaaggttatcttaattgaagccctgcctaatatcctaaacatgttcgataagacgttgatcaagtatgccgaggacctttttgccagagatgaaattgacttgcaagtgaatactgccgtgaaagtcgtagagccaacctatatacgcactctgcaaaacggccaaacaaacacggatatcgaatacgggatgctggtttgggccacgggaaatgaaccaatcgatttttcaaagacactgatgagtagaataccggagcaaactaataggcgtggtctgttaattaatgacaagttggagcttctcggttctgagaattcgatttatgcaattggtgattgtaccgcacacacgggtttctttcccacggcacaagttgcacatcaggaaggcgaatacttggccaagatcttggataaaaaattacagatagaacaattggaatgggacatgctcaacagtaccgatgaaactgaggtatcacgtctacaaaaagaggttaatttgaggaaatctaagttggataagttcaactacaagcatatgggtgcccttgcgtacatcggctctgaaaccgcaattgcagatttgcatatgggcgactcatcataccagttgaaaggtatgtttgccttcttgttttggaaatccgcttatttggccatgtgtctctctatcaggaataggattttaattgccatggactggaccaaagtttactttcttggaagggattcctccgtgtag

SEQ ID NO: 30 - DNA, 31 bp, Artificial Sequence
  Synthetic (forward primer)
cgagctcttcgcggccacctacgccgctatc

SEQ ID NO: 31 - DNA, 32 bp, Artificial Sequence
  Synthetic (reverse primer)
gctctagatattgatatagtgtttaagcgaat

SEQ ID NO: 32 - DNA, 26 bp, Artificial Sequence
  Synthetic (forward primer)
ggatccatgtccgtaaaggaactact

SEQ ID NO: 33 - DNA, 34 bp, Artificial Sequence
  Synthetic (reverse primer)
acgcgtcgacttaaaactgcaattccttttgaat

SEQ ID NO: 34 - DNA, 26 bp, Artificial Sequence
  Synthetic (forward primer)
gagctcaattaaccctcactaaaggg

SEQ ID NO: 35 - DNA, 26 bp, Artificial Sequence
  Synthetic (reverse primer)
gagctccaaattaaagccttcgagcg

SEQ ID NO: 36 - DNA, 69 bp, Artificial Sequence
  Synthetic (forward primer)
tgagcacgtgagtatacgtgattaagcacacaaaggcagcttggagtatggtgctgcaaggcgattaag

SEQ ID NO: 37 - DNA, 67 bp, Artificial Sequence
  Synthetic (reverse primer)
aggcaagtgcacaaacaatacttaaataaatactactcagtaataacccggctcgtatgttgtgtgg

SEQ ID NO: 38 - DNA, 21 bp, Artificial Sequence
  Synthetic (forward primer)
gccaaatgatttagcattatc

SEQ ID NO: 39 - DNA, 21 bp, Artificial Sequence
  Synthetic (reverse primer)
aaaaggagagggccaagaggg

SEQ ID NO: 40 - DNA, 60 bp, Artificial Sequence
  Synthetic (forward primer)
atgattagacaatcattaatgaaaacagtgtgggctaactccagtcacgacgttgtaaaa

SEQ ID NO: 41 - DNA, 60 bp, Artificial Sequence
  Synthetic (reverse primer)
ctagatagatgaatctctacccaagaaataaactttagccaggtttcccgactggaaagc

SEQ ID NO: 42 - DNA, 22 bp, Artificial Sequence
  Synthetic (forward primer)
actgatcatcatttaaaaatgt

SEQ ID NO: 43 - DNA, 22 bp, Artificial Sequence
  Synthetic (reverse primer)
aaggaaaaaaattttcacacta

SEQ ID NO: 44 - DNA, 70 bp, Artificial Sequence
  Synthetic (forward primer)
atgctgcccagacttggttttgcgaggactgctaggtccatacaccgtttccagtcacgacgttgtaaaa

SEQ ID NO: 45 - DNA, 60 bp, Artificial Sequence
  Synthetic (reverse primer)
ctacacggaggaatcccttccaagaaagtaaactttggtcaggtttcccgactggaaagc

SEQ ID NO: 46 - DNA, 20 bp, Artificial Sequence
  Synthetic (forward primer)
caggaacatagtagaaagac

SEQ ID NO: 47 - DNA, 18 bp, Artificial Sequence
  Synthetic (reverse primer)
taacgcgaatcttccatg

SEQ ID NO: 48 - DNA, 65 bp, Artificial Sequence
  Synthetic (forward primer)
aatgtctcaaccggttcaacgcgctgcagcacgctcattccttcaaagagctgcaccgcggtggc

SEQ ID NO: 49 - DNA, 65 bp, Artificial Sequence
  Synthetic (reverse primer)
ttactgtttaccagttttttctttctctttccattccttatctagagaccgggccccccgctagt

SEQ ID NO: 50 - DNA, 64 bp, Artificial Sequence
  Synthetic (forward primer)
aatgtctacatcatccgtacgttttgcatttaggcggttctggcaagagctgcaccgcggtggc

SEQ ID NO: 51 - DNA, 66 bp, Artificial Sequence
  Synthetic (reverse primer)
ttatctgcccgtagtaatttcctttttgctttctgcggcgccgctgagaccgggccccccgctagt

SEQ ID NO: 52 - DNA, 19 bp, Artificial Sequence
  Synthetic (forward primer)
ggacgtggcctgtaaagtt

SEQ ID NO: 53 - DNA, 23 bp, Artificial Sequence
  Synthetic (reverse primer)
agaatatgcatgaacatatccat

SEQ ID NO: 54 - DNA, 18 bp, Artificial Sequence
  Synthetic (forward primer)
ctattgcgcgcatgacta

SEQ ID NO: 55 - DNA, 20 bp, Artificial Sequence
  Synthetic (reverse primer)
tgcattgccttctattatcc

(19) United States
(12) Patent Application Publication
(10) Pub. No.: US 2015/0087032 A1
(43) Pub. Date: Mar. 26, 2015

(54) YEAST CELL WITH INCREASED PYRUVATE POOL IN CYTOSOL AND METHOD OF PRODUCING PYRUVATE-BASED METABOLITE USING THE SAME

(71) Applicant: Samsung Electronics Co., Ltd., Suwon-si (KR)

(72) Inventors: Young-kyoung PARK, Seoul (KR); Chang-duk Kang, Gwacheon-si (KR); Ji-yoon Song, Seoul (KR); Ju-young Lee, Daegu (KR); Seung-hyun Lee, Asan-si (KR); Kwang-myung Cho, Seongnam-si (KR)

(21) Appl. No.: 14/336,945

(22) Filed: Jul. 21, 2014

(30) Foreign Application Priority Data
Sep. 25, 2013 (KR) 10-2013-0114144

(51) Int. Cl. C12P 7/56; C12P 7/06

(52) U.S. Cl. CPC C12P 7/56; C12P 7/06
USPC 435/116; 435/254.21; 435/139; 435/161; 435/159; 435/140; 435/136; 435/171; 435/168

(57) ABSTRACT

A genetically engineered yeast cell that produces a pyruvate-based metabolite from pyruvate, wherein activity of a mitochondrial pyruvate carrier (MPC) is reduced compared to a parent yeast cell and a method of producing the pyruvate-based metabolite using the yeast cell.

### CROSS-REFERENCE TO RELATED APPLICATION

This application claims the benefit of Korean Patent Application No. 10-2013-0114144, filed on Sep. 25, 2013 in the Korean Intellectual Property Office, the entire disclosure of which is incorporated herein by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 69,836 Bytes ASCII (Text) file named “716701_ST25.TXT,” created on Jul. 16, 2014.

### BACKGROUND

1. Field

The present disclosure relates to yeast cells that produce a pyruvate-based metabolite from pyruvate in which activity of a mitochondrial pyruvate carrier (MPC) is reduced compared to yeast cells that are not genetically engineered, and methods of producing a pyruvate-based metabolite by using the yeast cells.

2. Description of the Related Art

Microorganisms such as bacteria or yeasts can produce useful materials via a biological fermentation process. The biological fermentation process requires optimization of various intracellular metabolic pathways to produce a target material at a high yield.

Pyruvate is an end product of a glycolysis process within a cell. Pyruvate is a major substrate used in the tricarboxylic acid (TCA) cycle in mitochondria and is converted into a variety of metabolites. In some cases, pyruvate may be converted into metabolites within the TCA cycle or may be converted into lactate. In some other cases, pyruvate may be converted into diacetyl, acetoin, or butanediol via α-acetolactate. In still other cases, pyruvate may be converted into acetaldehyde, ethanol, or acetate via acetyl-CoA. In this regard, pyruvate is involved in the production of various intracellular metabolites, and accordingly, in order to maximize production of a target pyruvate-based metabolite by using a biotechnological method, there is a need to appropriately adjust one or more metabolic pathways in which pyruvate is involved, or adjust the availability of intracellular pyruvate.

Lactate, which is an example of the pyruvate-based metabolite, is an organic acid that is widely used in various industrial fields, such as those of food, pharmaceuticals, chemicals, and electronics. Lactate is non-toxic to the human body, and thus may be used as a flavor agent, a taste agent, or a preserving agent. Also, lactate is a raw material of polylactic acid (PLA) which is an environmentally-friendly biodegradable plastic (i.e. polymer material). In addition, lactate includes both a hydroxyl group and a carboxyl group, and thus is highly reactive. Accordingly, lactate may be easily converted into industrially important compounds, such as lactate ester, acetaldehyde, and propylene glycol, and thus has received attention as an alternative next generation chemical material in the chemical industry.

Lactate is currently produced by either a chemical synthesis process or a biochemical process. The chemical synthesis process is performed by oxidizing ethylene derived from crude oil, preparing lactonitrile through addition of hydrogen cyanide after acetaldehyde, purifying by distillation, and hydrolyzing by using hydrochloric acid or sulfuric acid. The biochemical process converts pyruvate to lactate via the enzyme lactate dehydrogenase. Also, lactate is produced commercially by incubating microorganisms such as bacteria and yeasts with an assimilable carbohydrate substrate, such as starch, sucrose, maltose, glucose, fructose, and xylose.

Also, ethanol, which is another example of a pyruvate-based metabolite, is a material that is converted from pyruvate by pyruvate decarboxylase (PDC) and alcohol dehydrogenase (ADH) within a cell, and such a material is produced commercially using a fermentation method for microorganisms such as yeasts that produce ethanol. Ethanol is used in various applications related to alcoholic beverages and fuels.

Therefore, a strain for efficiently producing a target pyruvate-based metabolite from pyruvate, and a method of producing a pyruvate-based metabolite using the strain have been demanded.

### SUMMARY

Provided are genetically engineered yeast cells that produce a pyruvate-based metabolite from pyruvate, wherein the genetically engineered yeast cell has a deletion or disruption mutation of a gene encoding MPC and the activity of a mitochondrial pyruvate carrier (MPC) protein is reduced in comparison with parent yeast cells not having a deletion or disruption mutation of the gene encoding MPC.

Also provided are methods of preparing the genetically engineered yeast cells, and methods of producing a pyruvate-based metabolite using the genetically engineered yeast cells.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1 is a diagram illustrating a lactate production pathway of a yeast cell having the ability to produce lactate;

FIG. 2 is a diagram of a p416-CCW12p-LDH vector;

FIG. 3 is a diagram of a pUC57-ura3HA vector;

FIG. 4 is a diagram of a pUC57-CCW12-LDH-ura3HA vector;

FIG. 5 is a diagram of a pUC19-HIS3 vector;

FIG. 6 is a diagram of a pUC19-CCW12p-LDH-HIS3 vector; and

FIGS. 7A and 7B are each a graph showing culturing characteristics of strains of Δnde1 Δnde2 and Δnde1Δnde2Δmpc1Δmpc2 under fermentation conditions.

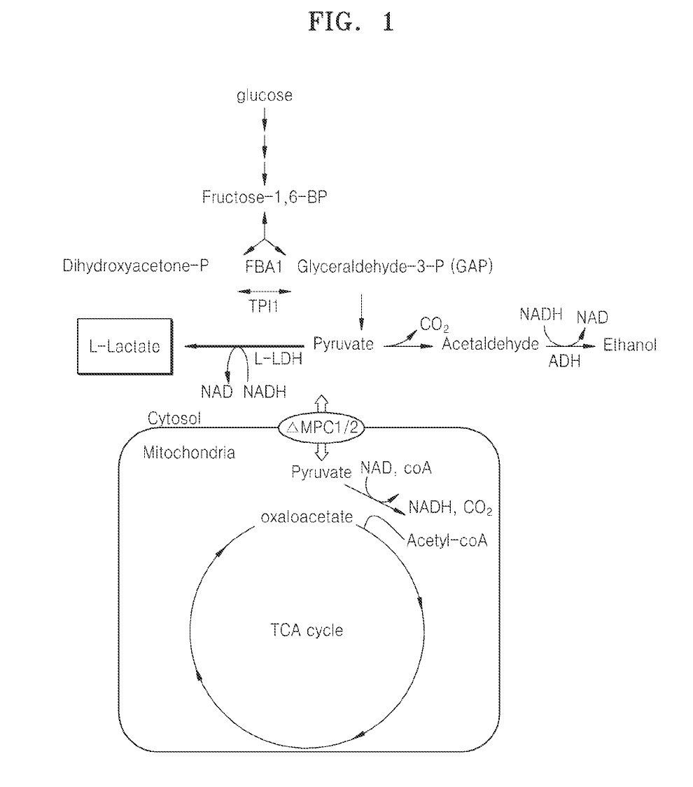

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

As used herein, the term “reduced activity” of a genetically engineered cell, enzyme or polypeptide denotes a genetically engineered cell, polypeptide or enzyme within a cell, or an isolated polypeptide or enzyme whose activity of a given type is lower than the activity measured in a comparable parent cell, polypeptide, or enzyme (e.g., the original or wild-type cell, polypeptide. or enzyme). The terms “activity reduces” or “reduced activity” of a cell, enzyme or polypeptide may also denote a cell, an isolated polypeptide or enzyme, or a polypeptide or enzyme within a cell having no activity of a given type. The activity of a cell, polypeptide, or enzyme may be reduced by any amount, e.g., about 20% or more, about 30% or more, about 40% or more, about 50% or more, about 55% or more, about 60% or more, about 70% or more, about 75% or more, about 80% or more, about 85% or more, about 90% or more, about 95% or more, or about 100% in comparison to the activity of a parent cell, wild-type polypeptide, or wild-type enzyme. The reduced activity of the cell (e.g., yeast), polypeptide, or enzyme may be confirmed using a commonly known method in the art. The reduction of the activity may be due to reduced expression of the gene, or reduced activity of the expressed polypeptide (e.g., a mutant polypeptide). The term “parent cell” denotes a cell not having a specific genetic modification resulting in a genetically engineered cell. The term “wild-type” polypeptide or polynucleotide denotes a polypeptide or polynucleotide not having a specific genetic modification resulting in a genetically engineered polypeptide or polynucleotide.

Activity of a cell, polypeptide, or enzyme may be reduced due to deletion or disruption of a gene encoding the polypeptide or enzyme (e.g., which a cell). As used herein, the “deletion” or “disruption” of the gene includes mutation or deletion of the gene or a regulatory region of the gene (e.g., operator, promoter or terminator regions of the gene), or a part thereof, sufficient to disrupt or delete gene function or the expression of a functional gene product. Mutations include substitutions, additions, and deletions of one or more bases in the gene or its regulator regions. As a result, the gene is not expressed, or the gene has a reduced amount of expression, or the activity of the encoded protein or enzyme is reduced or eliminated. The deletion or disruption of the gene may be accomplished by any suitable genetic engineering technique, such as homologous recombination, mutation induction, or molecular evolution. When a cell includes a plurality of copies of the same gene or at least two different polypeptide paralogs, at least one gene may be deleted or disrupted.

As used herein, the term “increased activity” of a genetically engineered cell, enzyme or polypeptide means that the genetically engineered cell, a polypeptide or enzyme within a cell, or an isolated polypeptide or enzyme exhibits an activity level of a given type that is higher than the activity level of a comparable parent cell, wild-type polypeptide, or wild-type enzyme. The activity of a cell, polypeptide, or enzyme may be increased by any amount, e.g., by about 5% or more, about 10% or more, about 15% or more, about 20% or more, about 30% or more, about 50% or more, about 60% or more, about 70% or more, or about 100% or more in comparison to the activity of a parent cell, wild-type polypeptide, or wild-type enzyme. A cell having increased activity of a cell, polypeptide, or enzyme may be confirmed by using any method commonly known in the art.

An increase in the activity of the cell, polypeptide, or enzyme may be caused by an increase in expression of the polypeptide or enzyme (e.g., within a cell) or an increase in a specific activity of the polypeptide or enzyme (e.g., within a cell). The increase in the expression may be caused by introduction of one or more polynucleotides encoding a polypeptide or enzyme into a cell, thereby introducing a new polynucleotide into the cell or providing an increased number of copies of an existing polynucleotide, by mutation of an expressed sequence to provide a mutant gene product having enhanced activity, or by mutation of a regulatory region of the polynucleotide so as to increase expression. Here, the introduced polynucleotide or the polynucleotide having the increased number of copies may be an endogenous gene or an exogenous gene. The endogenous gene may be a gene present in a genetic material contained within the microorganism. The exogenous gene may include a gene not normally occurring in the host cell that is integrated into the genetic material of the host cell (e.g., a chromosome of a host cell) or a gene introduced into the host cell by way of expression vector (e.g., a plasmid) or other construct.

As used herein, the gene manipulation and engineering may be performed by molecular biological methods known in the art (refer to Roslyn M. Bill, Recombinant Protein Production in Yeast: Methods and Protocols (2012), Sambrook et al., Molecular cloning, A laboratory manual: Cold Spring Harbor Laboratory (1989), R Daniel Gietz et al., Quick and easy yeast transformation using the LiAc/SS carrier DNA/PEG method: Nature protocols (2007)).

As used herein, the term “gene” denotes a nucleic acid fragment expressing a specific protein, and the fragment may include a coding region as well as non-coding regions or regulatory sequences (e.g., 5′-non coding sequences and a 3′-non coding sequences) in addition to the coding region.

As used herein, the term “pyruvate-based metabolite” or “pyruvate metabolite” denotes an intracellular metabolite converted from pyruvate. A pyruvate-based metabolite of the present disclosure may be a metabolite converted from pyruvate within a cytosol, or a metabolite of a pyruvate-based metabolite precursor that is converted from pyruvate in a cytosol. The pyruvate-based metabolite may include a metabolite directly converted from pyruvate by a biosynthetic step or a metabolite converted from the metabolite directly converted from pyruvate pyruvate by a biosynthetic step. The pyruvate-based metabolite may be at least one selected from lactate, ethanol, glycerol, acetate, formate, alanine, carbon dioxide, hydrogen, and a combination thereof. When the pyruvate-based metabolite is an organic acid, the pyruvate-based metabolite may include free organic acid, anionic form, or a salt thereof. Thus, free organic acid may be interchangeably used with its ‘anionic form’ or a salt thereof. For example, lactic acid may be interchangeably used with lactate or a salt thereof. The “pyruvate-based metabolite” may be secreted from the cell to the outside of the cell.

As used herein, the term “sequence identity” of a nucleic acid or polypeptide with respect to another nucleic acid or polypeptide refers to a degree of similarity in the sequence of bases or amino acid residues of two sequences (or a given region of two sequences) that are aligned to best match each other for comparison. The sequence identity is a value obtained via optimal alignment and comparison of the two sequences in the specific region for comparison, in which a partial sequence in the specific region for comparison may be added or deleted with respect to a reference sequence. The sequence identity represented in a percentage may be calculated by, for example, comparing two sequences that are aligned to best match each other in the specific region for comparison, determining matched sites, wherein the same amino acid or base in the two sequences is found, to obtain the number of the matched sites, dividing the number of the matched sites in the two sequences by a total number of sites in the compared specific regions (i.e., the size of the compared region), and multiplying a result of the division by 100 to obtain a sequence identity as a percentage. The sequence identity as a percentage may be determined using a known sequence comparison program, for example, BLASTN or BLASTP (NCBI), CLC Main Workbench (CLC bio), MegAlign™ (DNASTAR Inc), or the like.

In identifying a polypeptide or polynucleotide with the same or similar function or activity with respect to various types of species, various levels of sequence identity may be applied. In some embodiments, the polypeptide or polynucleotide may have an amino acid sequence, or a nucleic acid sequence, respectively, with a sequence identity of, for example, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, 99% or greater, or 100%, with respect to the other polypeptide or polynucleotide to which it was compared.

According to one aspect of the present disclosure, provided is a genetically engineered yeast cell that produces a pyruvate-based metabolite from pyruvate, wherein the genetically engineered yeast cell has a deletion or disruption mutation of a gene encoding MPC and activity of a mitochondrial pyruvate carrier (MPC) is reduced compared to a parent yeast cell not having a deletion or disruption mutation of the gene encoding MPC.

According to another aspect of the present disclosure, there is provided a method of producing a pyruvate-based metabolite from pyruvate using a yeast cell by reducing the activity of one or more MPC proteins in the yeast cell.

Production of the pyruvate-based metabolite from pyruvate may occur in a cytosol.

In regard to the production of the pyruvate-based metabolite converted from pyruvate or a precursor of the pyruvate-based metabolite in a cytosol using a yeast cell, when the level of pyruvate in a cytosol is increased, an amount of the pyruvate-based metabolite produced from pyruvate or the pyruvate-based metabolite precursor may be increased. The level of pyruvate within a cytosol may be increased by the reduction of the activity of the MPC polypeptides.

In general, pyruvate is produced from glucose through glycolysis in the cytosol, and the produced pyruvate is delivered to a mitochondrion by one or more MPC proteins. The transported pyruvate is then converted into acetyl-CoA, and used to produce metabolites or energy via a tricarboxylic acid (TCA) cycle. When the activity of MPC is reduced in a cell and the delivery of pyruvate from the cytosol to the mitochondrion is inhibited, a pyruvate pool within the cytosol may be enhanced, and accordingly the production of the pyruvate-based metabolite may be increased therefrom.

The activity of the MPC may be reduced by deletion or disruption of the MPC-coding polynucleotide. The MPC is a polypeptide present in the inner mitochondrial membrane and acts to transport pyruvate from the cytosol to mitochondria. The MPC may be a MPC 1, a MPC2, a MPC3, and any one of these proteins, or a combination thereof, may be inhibited by deletion or disruption of the corresponding gene. For example, the MPC may have an amino acid sequence with a sequence identity of about 60% or greater, about 70% or greater, about 80% or greater, about 90% or greater, about 95% or greater, about 96% or greater, about 97% or greater, about 98% or greater, or about 99% or greater with respect to SEQ ID NOs: 1, 2, or 3. In greater detail, the MPC may have an amino acid sequence with a sequence identity of about 95% or greater with respect to SEQ ID NOs: 1, 2, or 3. The MPC 1, MPC2, and MPC3 may each have an amino acid sequence of SEQ ID NOs: 1, 2, and 3, respectively. The MPC-coding polynucleotide may be a polynucleotide encoding a protein having an amino acid sequence with a sequence identity of about 95% or greater with respect to SEQ ID NOs: 1, 2, or 3. For example, the MPC-coding polynucleotide may have a nucleotide sequence of SEQ ID NOs: 4, 5, or 6. The MPC-coding polynucleotide having nucleotide sequences of SEQ ID NOs: 4, 5, and 6 each encode a MPC1, a MPC2, and a MPC3, respectively.

The yeast cell may belong to the Saccharomyces genus, Candida genus, Shizosaccharomyces genus, Kluyveromyces genus, Pichia genus, Issachenkia genus, or Hansenula genus. In some embodiments, the yeast cell may belong to Saccharomyces genus, Candida genus, Kluyveromyces genus, or Issachenkia genus. The Saccharomyces genus may be, for example, S. cerevisiae, S. bayanus, S. boulardii, S. bulderi, S. cariocanus, S. cariocus, S. chevalieri, S. dairenensis, S. ellipsoideus, S. eubayanus, S. exiguus, S. florentinus, S. kluyveri, S. martiniae, S. monacensis, S. norbensis, S. paradoxus, S. pastorianus, S. spencerorum, S. turicensis, S. unisporus, S. uvarum, or S. zonatus.

The genetically engineered yeast cell of the present disclosure may have an increased ability (e.g., increased efficiency) to produce a pyruvate-based metabolite. The pyruvate-based metabolite may be at least one selected from lactate, ethanol, glycerol, acetate, formate, alanine, carbon dioxide, hydrogen, and a combination thereof. In some embodiments, the conversion of a pyruvate-based metabolite from pyruvate or from the pyruvate-based metabolite precursor may occur in the cytosol. The increased level of pyruvate in the cytosol due to the reduced activity of the MPC may facilitate the conversion of the pyruvate-based metabolite from pyruvate.

The genetically engineered yeast cell may be produced from a strain that produces a pyruvate-based metabolite naturally, whereby the genetic engineering described herein enhances the production of the metabolite. Alternatively, the genetically engineered yeast cell may be produced from a strain that does not produce a pyruvate-based metabolite naturally. When the yeast cell may not produce a pyruvate-based metabolite naturally (e.g., when the yeast cell does not contain the endogenous genes necessary to produce the specific pyruvate-based metabolite), the genes involved in the production of the pyruvate-based metabolite may be introduced into the yeast cell to produce the pyruvate-based metabolite. In either case, the yeast cell in which activity of the MPC is reduced may increase the production of the pyruvate-based metabolite in comparison to the cell that is not genetically engineered to reduce activity of the MPC.

In some embodiments, the yeast cell may have an ability to produce lactate. The yeast cell may include a polypeptide converting pyruvate into lactate, such as a lactate dehydrogenase (LDH). In some other embodiments, the yeast cell may be genetically engineered to have an increased activity of the polypeptide converting pyruvate into lactate as compared to a parent cell. The LDH may be an NAD(P)-dependent enzyme or a stereo-specific enzyme. The stereo-specific enzyme may produce L-lactate, D-lactate, or a combination thereof. The NAD(P)-dependent enzyme may be an enzyme that is classified into EC 1.1.1.27 that functions on L-lactate or EC 1.1.1.28 that functions on D-lactate.

The yeast cell having an ability to produce lactate may be genetically engineered to have increased LDH activity as compared to a parent cell. The yeast cell used herein may include at least one LDH gene, and the LDH gene may be an endogenous gene or an exogenous gene. The polynucleotide encoding the LDH may be an enzyme derived from bacteria, yeasts, fungi, mammals or reptiles. The polynucleotide may be a polynucleotide that encodes at least one LDH from at least one of Lactobacillus helveticus, L. bulgaricus, L. johnsonii, L. plantarum, Pelodiscus sinensis japonicus, Ornithorhynchus anatinus, Tursiops truncatus, Rattus norvegicus, and Xenopus laevis. An LDH from Pelodiscus sinensis japonicus, an LDH from Ornithorhynchus anatinus, an LDH from Tursiops truncatus, and an LDH from Rattus norvegicus may each have an amino acid sequence with a sequence identity of about 60% or greater, about 70% or greater, about 80% or greater, about 90% or greater, about 95% or greater, about 96% or greater, about 97% or greater, about 98% or greater, or about 99% or greater with respect to SEQ ID NOs: 7, 8, 9, and 10. In some embodiments, the LDH-coding polynucleotide may be a polynucleotide encoding an amino acid sequence with a sequence identity of about 95% or greater with respect to SEQ ID NOs: 7, 8, 9, and 10. In some other embodiments, the LDH-coding polynucleotide may be a polynucleotide having a nucleotide sequence with a sequence identity of about 95% or greater with respect to SEQ ID NO: 11.

The LDH-coding polynucleotide may be contained in a vector, and the vector may include a replication origin, a promoter, a LDH-coding polynucleotide, and a terminator. The replication origin may include a yeast autonomous replication sequence (ARS). The yeast ARS may be stabilized by a yeast centrometric sequence (CEN). The promoter may be selected from a cytochrome c (CYC) promoter, a transcription elongation factor (TEF) promoter, a glycerol-3-phosphate dehydrogenase (GPD) promoter, and an alcohol dehydrogenase (ADH) promoter. The CYC promoter, the TEF promoter, the GPD promoter, and the ADH promoter may each have a nucleotide sequence of SEQ ID NOs: 13, 14, 15, and 16.

The terminator may be selected from a terminator of a gene encoding a phosphoglycerate kinase 1 (PGK1), a terminator of a gene encoding a cytochrome c 1 (CYC1), and a terminator of a gene encoding a galactokinase 1 (GAL1). The CYC1 terminator may have a nucleotide sequence of SEQ ID NO: 17.

The vector may further include a selection marker. The selection marker may any known selection marker in the art. For example, the selection marker may be auxotrophic marker, or antibiotic resistance selection marker.

The LDH-coding polynucleotide may be included in a genome in a specific location of a yeast cell. The specific location of the yeast cell may include a locus of a gene to be deleted and disrupted, such as MPC, pyruvate decarboxylase (PDC) and cytochrome-c oxidoreductase 2 (CYB2).

The yeast cell may include a polynucleotide that encodes one LDH or a polynucleotide that encodes multiple LHD copies, e.g., 2 to 10 copies. The yeast cell may include a polynucleotide that encodes multiple LDH copies into, for example, 1 to 8, 1 to 7, 1 to 6, 1 to 5, 1 to 4, or 1 to 3 copies. When the yeast cell includes the polynucleotide encoding multiple LDHs, each polynucleotide may include copies of the same LDH polynucleotide or copies of polynucleotides encoding at least two different LDHs. The multiple copies of the polynucleotide encoding exogenous LDHs may be included in the same locus or multiple loci in a genome of a host cell, and the promoter or terminator of each copy of the polynucleotide may be identical to or different from each other.

In order to optimize the production of the pyruvate-based metabolite, a metabolic pathway of the yeast cell may be optimized. That is, activity of a metabolic pathway which is a competitive pathway with the production of the pyruvate-based metabolite may be reduced, or activity of a metabolic pathway which is a synergistic pathway with the production of the pyruvate-based metabolite may be increased.

In some embodiments, the yeast cell used herein may exhibit lactate productivity and may further include genetic modification to optimize lactate production. For example, in the yeast cell, activity of a polypeptide that converts pyruvate into acetaldehyde, a polypeptide that converts lactate into pyruvate, a polypeptide that converts dihydroxyacetone phosphate (DHAP) into glycerol-3-phosphate, or a combination thereof may be reduced.

In the genetically engineered yeast cell, a gene that encodes the polypeptide that converts pyruvate into acetaldehyde may be deleted or disrupted. The polypeptide that converts pyruvate into acetaldehyde may be an enzyme that is classified as EC 4.1.1.1. In some embodiments, the enzyme may be a pyruvate decarboxylase (PDC), for example, a PDC1. The polypeptide that converts pyruvate to acetaldehyde may have an amino acid sequence with a sequence identity of about 60% or greater, about 70% or greater, about 80% or greater, about 90% or greater, about 95% or greater, about 96% or greater, about 97% or greater, about 98% or greater, or about 99% or greater with respect to SEQ ID NO 18. The gene that encodes the polypeptide that converts pyruvate to acetaldehyde may be a polynucleotide encoding an amino acid sequence with a sequence identity of about 95% or greater with respect to SEQ ID NO 18, or may have a nucleotide sequence of SEQ ID NO: 19.

In the yeast cell, a gene that encodes the polypeptide that converts lactate into pyruvate may be disrupted. The polypeptide that converts lactate into pyruvate may be a CYC-dependent enzyme. The polypeptide that converts lactate into pyruvate may be an enzyme that is classified as EC 1.1.2.4 that acts on D-lactate or EC 1.1.2.3 that acts on L-lactate. The polypeptide that converts lactate into pyruvate may be lactate cytochrome c-oxidoreductase, a CYB2 (CAA86721.1), a CYB2A, a CYB2B, or a DLD1. The polypeptide that converts lactate into pyruvate may have an amino acid sequence with a sequence identity of about 60% or greater, about 70% or greater, about 80% or greater, about 90% or greater, about 95% or greater, about 96% or greater, about 97% or greater, about 98% or greater, or about 99% or greater with respect to SEQ ID NO 20. The gene that encodes the polypeptide that converts lactate into pyruvate may be a polynucleotide encoding an amino acid sequence with a sequence identity of about 95% or greater with respect to SEQ ID NO 20, or may have a nucleotide sequence of SEQ ID NO: 21.

In the yeast cell, a gene that encodes the polypeptide that converts DHAP into glycerol-3-phosphate may be disrupted. The polypeptide may be glycerol-3-phosphate dehydrogenase (GPD), which is an enzyme that catalyzes reduction of DHAP to glycerol-3-phosphate by using oxidation of NADH or NADP to NAD+ or NADP+. The polypeptide may belong to EC 1.1.1.8, and the cytosolic GPD may be GPD1. The cytosolic GPD may have an amino acid sequence with a sequence identity of about 60% or greater, about 70% or greater, about 80% or greater, about 90% or greater, about 95% or greater, about 96% or greater, about 97% or greater, about 98% or greater, or about 99% or greater with respect to SEQ ID NO 22. The gene that encodes cytosolic GPD may a polynucleotide encoding an amino acid sequence with a sequence identity of about 95% or greater with respect to SEQ ID NO 22, or may have a nucleotide sequence of SEQ ID NO: 23.

In some embodiments, in the yeast cell, the polynucleotide that encodes the MPC, the polynucleotide that encodes the polypeptide converting pyruvate into acetaldehyde, the polynucleotide that encodes the polypeptide converting lactate into pyruvate, the polynucleotide that encodes the polypeptide converting DHAP into glycerol-3-phosphate, or a combination thereof may be disrupted, and in addition, a polynucleotide that encodes a polypeptide converting pyruvate into lactate may be included or further introduced into the yeast cell. Here, the yeast cell may be Saccharomyces cerevisiae.

In some embodiments, the pyruvate-based metabolite may include ethanol, and thus the genetically engineered yeast cell may produce ethanol. In the yeast cell that may produce ethanol, activity of converting pyruvate into ethanol may be increased. In addition, the yeast producing ethanol may include PDC, which is an enzyme catalyzing conversion from pyruvate into acetaldehyde, and alcohol dehydrogenase (ADH), which is an enzyme catalyzing conversion from acetaldehyde into ethanol.

The PDC may be an enzyme that is classified as EC 4.1.1.1, and the enzyme may be a PDC1. The PDC may have an amino acid sequence with a sequence identity of about 60% or greater, about 70% or greater, about 80% or greater, about 90% or greater, about 95% or greater, about 96% or greater, about 97% or greater, about 98% or greater, or about 99% or greater with respect to SEQ ID NO 18. The gene that encodes the PDC may be a polynucleotide encoding an amino acid sequence with a sequence identity of about 95% or greater with respect to SEQ ID NO 18, or may have a nucleotide sequence of SEQ ID NO: 19.

The ADH may be an enzyme that is classified as EC 1.1.1.1, and the enzyme may be an ADH1. The ADH may have an amino acid sequence with a sequence identity of about 60% or greater, about 70% or greater, about 80% or greater, about 90% or greater, about 95% or greater, about 96% or greater, about 97% or greater, about 98% or greater, or about 99% or greater with respect to SEQ ID NO 24. The gene that encodes the ADH may be a polynucleotide encoding an amino acid sequence with a sequence identity of about 95% or greater with respect to SEQ ID NO 24, or may have a nucleotide sequence of SEQ ID NO: 25.

In some embodiments, the yeast cell used herein may have an ability to produce ethanol and may further include genetic modification to optimize the production of ethanol. For example, in the yeast cell, activity of a polypeptide that converts pyruvate into lactate, a polypeptide that converts DHAP into glycerol-3-phosphate, or a combination thereof may be reduced. The polypeptide may be the same as described above.

In some other embodiments, in the yeast cell, the polynucleotide that encodes the MPC, the polynucleotide that encodes the polypeptide converting pyruvate into lactate, the polynucleotide that encodes the polypeptide converting DHAP into glycerol-3-phosphate, or a combination thereof may be disrupted, and in addition, a polynucleotide that encodes a polypeptide converting pyruvate into ethanol may be included or further introduced into the yeast cell. Here, the yeast cell may be Saccharomyces cerevisiae.

In some embodiments, the pyruvate-based metabolite may include glycerol, acetate, formate, alanine, carbon dioxide, or hydrogen, and may be produced by using a known biosynthetic pathway or enzyme in the art (Appl Environ Microbiol. 2009 April; 75(7): 1867-875; Indian Journal of Microbiology, Volume 48, Issue 2, pp 252-266; Biotechnol Adv. 2001 June; 19(3):201-23).

In some embodiments, the yeast cell in which the activity of the MPC is reduced may be a yeast cell in which activity of a mitochondrial NADH dehydrogenase is further reduced. The external mitochondrial NADH dehydrogenase may be an enzyme that is classified as EC. 1.6.5.9 or EC. 1.6.5.3. The NADH dehydrogenase may be a type II NADH: ubiquinone oxidoreductase. The NADH dehydrogenase may be located on the outer surface of the inner mitochondrial membrane facing a cytoplasm. The NADH dehydrogenase may be an enzyme catalyzing oxidation of cytosolic NADH to NAD+. The NADH dehydrogenase may re-oxidize cytosolic NADH formed by a glycolysis process. The NADH dehydrogenase may provide cytosolic NADH to a mitochondrial respiratory chain. The NADH dehydrogenase may be Nde1, Nde2, or a combination thereof. The NADH dehydrogenase may be distinguished from an internal mitochondrial NADH dehydrogenase NDI1 that is present and functions inside mitochondria. The NADH dehydrogenase may have an amino acid sequence with a sequence identity of about 60% or greater, about 70% or greater, about 80% or greater, about 90% or greater, about 95% or greater, about 96% or greater, about 97% or greater, about 98% or greater, or about 99% or greater with respect to SEQ ID NOs: 26 or 27. For example, NDE1 and NDE2 may each have amino acid sequences of SEQ ID NO: 26 and SEQ ID NO: 27.

The genes encoding the external mitochondrial NADH dehydrogenase may each have an amino acid sequence with a sequence identity of about 95% or greater with respect to SEQ ID NOs:26 and 27, or may have a polynucleotide sequence of SEQ ID NOs: 28 or 29.

The method of producing a pyruvate-based metabolite comprises culturing the yeast cell, and collecting the pyruvate-based metabolite from the yeast culture.

The culturing of the yeast cell may be performed in a medium containing an assimilable-carbon source, for example, glucose. The medium used in the culturing of the yeast cell may be a common medium appropriate for growth of a host cell, a minimum medium or a composite medium containing an appropriate supplement.

The medium used in the culturing process may be a medium that may satisfy requirements of a particular yeast cell. The medium may be one selected from a carbon source, a nitrogen source, salts, trace elements, and a combination thereof.

Culture conditions may be appropriately controlled in the genetically engineered yeast cell to obtain a pyruvate-based metabolite. The culturing may be conducted in conditions suitable for the production of a pyruvate-based metabolite. The yeast cell may be cultured under aerobic conditions or microaerobic or anaerobic conditions. In some embodiments, the yeast cell may be cultured under aerobic conditions for its proliferation, and accordingly under anaerobic conditions to produce a pyruvate-based metabolite. The oxygen conditions may include a dissolved oxygen (DO) concentration of 0% to 10%, for example, 0%, 0 to 8%, 0 to 6%, 0 to 4%, 0 to 2%, 1 to 10%, 1 to 8%, 1 to 6%, 1 to 4%, or 1 to 2%, 2 to 10%, 2 to 8%, 2 to 6%, 2 to 4%, 3 to 10%, 3 to 8%, 3 to 6%, 4 to 10%, 4 to 8%, or 4 to 6%. Fermented broth may be controlled to maintain its pH in a range of about 2 to about 7.

The yeast cell used herein may be incubated by methods of continuous culture, semicontinuous culture, batch culture, fed batch culture, or a combination thereof.

As used herein, the term “culture condition” denotes a condition for culturing a yeast cell. The culture condition may be, for example, a carbon source, a nitrogen source, or oxygen conditions for the yeast cell. The carbon source that is used by the yeast cell includes monosaccharides, disaccharides, or polysaccharides. In particular, glucose, fructose, mannose, or galactose may be used. The nitrogen source that is used by the yeast cell may be an organic nitrogen compound or an inorganic nitrogen compound. In particular, amino acid, amide, amine, nitrate, or ammonium salt may be used. The oxygen condition for culturing the yeast cell may be an aerobic condition of a normal oxygen partial pressure, a low-oxygen condition including 0.1% to 10% of oxygen in the atmosphere, or an anaerobic condition including no oxygen. A metabolic pathway may be corrected with a carbon source or a nitrogen source that may be actually used by a yeast cell.

The collecting of the pyruvate-based metabolite from the yeast culture may further include a process of isolating or separating the pyruvate-based metabolite from the yeast culture. Separation of the pyruvate-based metabolite from the culture may be performed by a separation method commonly known in the art. The separation method may be centrifugation, filtration, extraction, distillation, ion-exchange chromatography, or crystallization.

Hereinafter, the present disclosure will be described in further detail with reference to the following examples. These examples are for illustrative purposes only and are not intended to limit the scope of the invention.

### Example 1

### Preparation of Strain for Highly-Efficient Production of Lactate and Preparation of Expression Vector

In order to block a production pathway of ethanol and glycerol as main byproducts by using Saccharomyces cerevisiae CEN.PK2-1D (MATaura3-52; trp1-289; leu2-3,112; his3Δ1; MAL2-8^{c}; SUC2, EUROSCARF accession number: 30000B) as a lactate production strain, the following genes were disrupted: a pyruvate decarboxylase (pdc1) gene, which is a main enzyme of alcohol fermentation; a NAD-dependent glycerol-3-phosphate dehydrogenase (gpd1) gene, which is a main enzyme of glycerol biosynthesis; and a L-lactate cytochrome-c oxidoreductase 2 (cyb2) gene, which is a lactate degrading enzyme. A lactate dehydroxygenase gene was introduced into each position of the disrupted genes of Saccharomyces cerevisiae CEN.PK2-1D, thereby obtaining Saccharomyces cerevisiae CEN.PK2-1D Δpdc1::Idh Δcyb2::IdhΔgpd1::Idh (KCTC 12415BP).

1.1 Preparation of a L-LDH Overexpression Vector

A CCW12 promoter polymerase chain reaction (PCR) fragment obtained by performing a PCR with a genomic DNA of Saccharomyces cerevisiae CEN.PK2-1D as a template and using primers of SEQ ID NO: 30 and SEQ ID NO: 31 was digested with SacI and XbaI, and the resultant was inserted into a p416-GPD vector (refer to http://www.atcc.org/products/all/87360.aspx) digested with SacI and XbaI, thereby producing a p416-CCW12p vector suitable for overexpression of L-Idh.

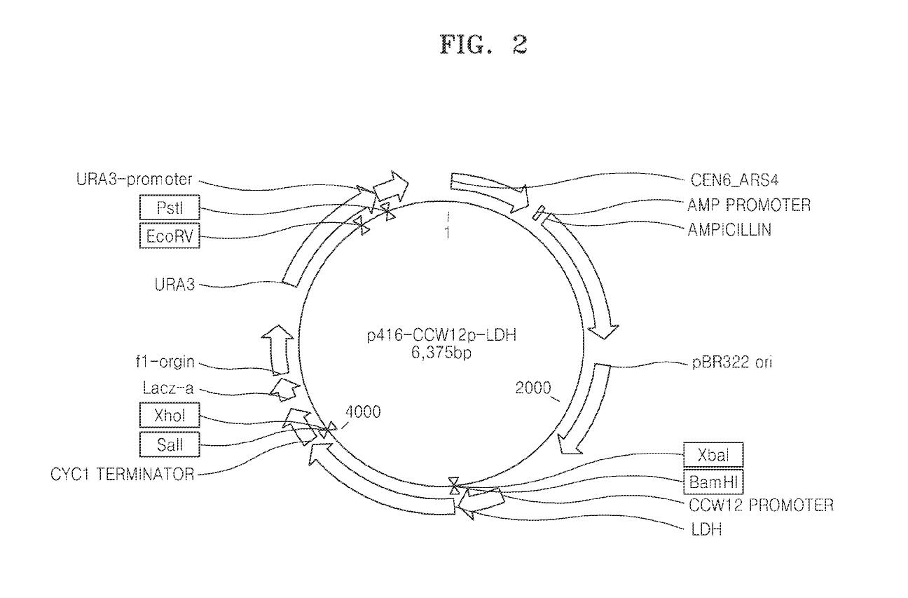

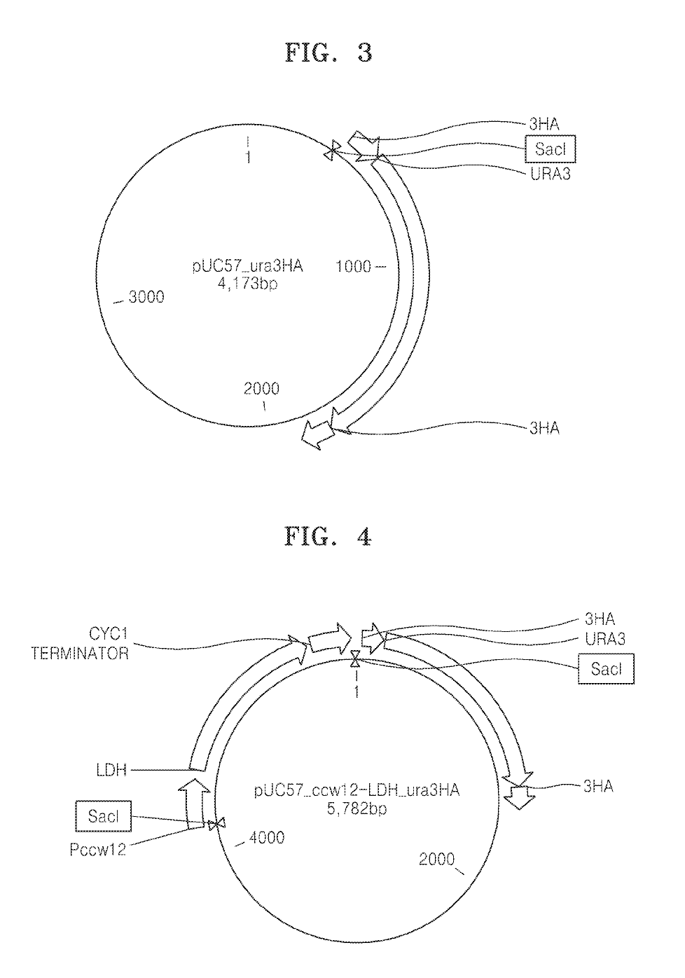

Then, L-Idh gene (SEQ ID NO: 11) was amplified from Pelodiscus sinensis japonicus genomic DNA by PCR using primers of SEQ ID NO: 32 and SEQ ID NO: 33. The resulting L-Idh PCR fragment and a p416-CCW12p obtained therefrom were digested with BamHI and SalI, and ligated, thereby producing p416-CCW12p-LDH, which is an L-Idh expression vector. The L-Idh expression vector also has a yeast autonomous replication sequence/a yeast centrometric sequence of SEQ ID NO: 12, a CYC promoter of SEQ ID NO: 13, a GPD promoter of SEQ ID NO: 15, and a CYC1 terminator of SEQ ID NO: 17. The L-Idh expression vector included a polynucleotide encoding L-Idh of SEQ ID NO: 11. FIG. 2 is a view illustrating a p416-CCW12p-LDH vector.

1.2 Preparation of Introduction Vector for Introducing L-Idh Gene into Genome

In order to increase lactate production by redox balance enhancement or glycolysis pathway engineering, an L-Idh gene may be additionally introduced to a genome of a KCTC12415BP strain. In order to introduce the L-Idh gene to the genome of the KCTC12415BP strain, a gene introduction vector may be prepared as follows.

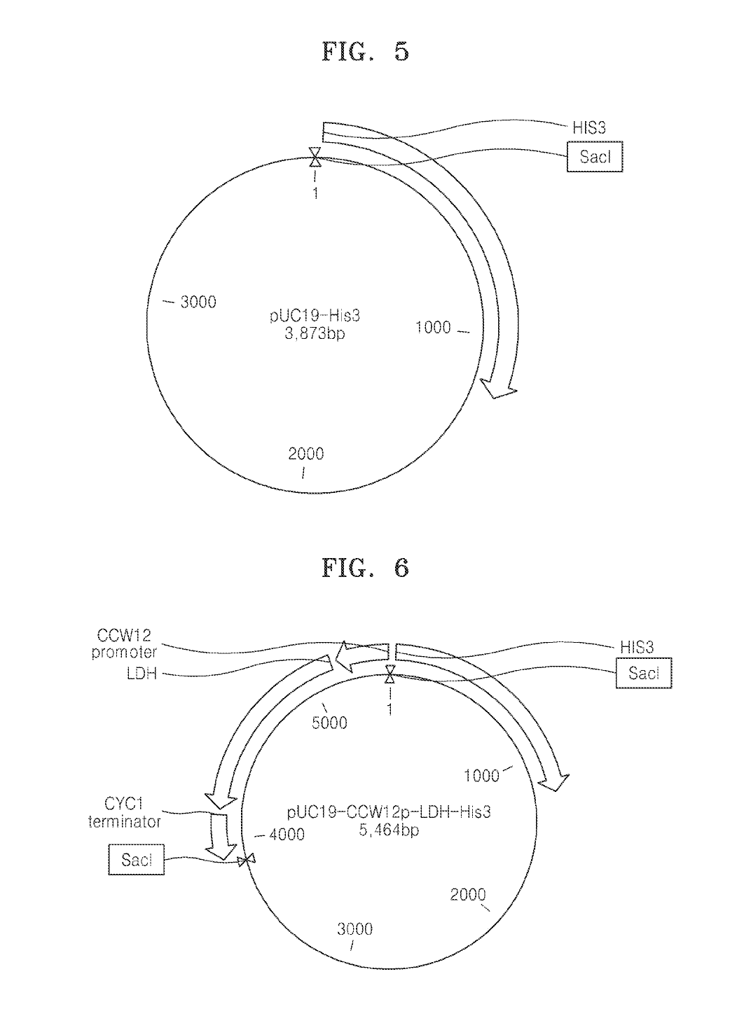

PCR was performed by using the prepared p416-CCW12p-LDH as a template with primers of SEQ ID NOs: 34 and 35. The resulting PCR fragment and the prepared pUC19-HIS3 vector (refer to Appl Environ Microbiol. 2002 May; 68(5):2095-100, and FIG. 5) were cleaved by SacI, and the resultant was ligated to prepare pUC19-CCW12p-LDH-HIS3 (see FIG. 6). PCR was performed by using the prepared pUC19-CCW12p-LDH-HIS3 as a template with primers of SEQ ID NOs: 36 and 37, and thus a cassette to be inserted in a location of TRP1 was prepared. The cassette including L-Idh may be inserted to a TRP1 gene, and in this case, L-Idh may be inserted as the TRP1 gene is deleted. An L-Idh inserted strain may be prepared as follows.

A strain of KCTC12415BP was spread onto a YPD agar plate (including 10 g of yeast extract, 20 g of peptone, and 20 g of glucose) and incubated for 24 hours at 30° C.° C., and then a colony obtained therefrom was inoculated in about 10 ml of a YPD liquid medium and cultured for 18 hours at 30° C.° C. The sufficiently grown culture solution was inoculated in about 50 ml of a YPD liquid medium contained in a 250 ml-flask at a concentration of 1% (v/v) and in an incubator vortexing at a rate of about 230 rpm and at 30° C.° C.

After about 4 to 5 hours, when the optical density at 600 nanometers (OD₆₀₀) reached about 0.5, the culture was centrifuged at a rate of about 4,500 rpm for about 10 minutes to harvest cells, and the cells were suspended in a lithium acetate solution at a concentration of about 100 mM. Then, the suspended cells were centrifuged at a rate of about 4,500 rpm for about 10 minutes to harvest cells, and the harvested cells were re-suspended in a lithium acetate solution at a concentration of about 1 M including about 15% of glycerol.

In order to delete a TRP1 gene and express L-Idh at the same time, a L-Idh expression cassette was mixed with 50% of polyethylene glycol and a single stranded carrier DNA, 100 ul of the re-suspension solution was added thereto, and reacted in a water tub at 42° C.° C. for 1 hour. Then, the culture solution was spread on a histidine (his)-free minimal agar plate (including YSD, 6.7 g/L of yeast nitrogen base without amino acids, and 1.4 g/L of amino acid dropout mix (-his)) and grown at 30° C.° C. for 24 hours or more. Ten colonies (i.e., mutant strains) grown on the his-free minimal agar plate were selected, spread onto a fresh YSD (-his) agar plate, and at the same time, inoculated into a YSD (-his) liquid medium to isolate the genomic DNA from the mutant strains above by using a commonly used kit (Gentra Puregene Cell kit, Qiagen, USA). In order to confirm deletion of the TRP1 gene, a PCR was performed using the isolated genomic DNA of the mutant strain as a template with primers of SEQ ID NOs: 38 and 39, and then, electrophoresis was performed on the obtained PCR product to confirm insertion of the L-Idh expression cassette. As a result, the obtained strain was Δtrp1::Idh (KCTC12415BP Δtrp1::Idh).

### Example 2

### Preparation of Saccharomyces cerevisiae Strain from which nde1 and nde2 are Deleted

2.1 Preparation of a nde1 Gene Deletion Cassette

Preparation of pUC57-CCW12p-LDH-ura3HA: a PCR was performed using the p416-CCW12p-LDH of Example 1 as a template with primers of SEQ ID NO: 34 and SEQ ID NO: 35. The resulting PCR fragment and the prepared pUC57-ura3HA vector (Genetics 116: 541-545, August, 1987) were digested with SacI and ligated, thereby producing pUC57-CCW12p-LDH-ura3HA.

A PCR was performed using the prepared pUC57-CCW12p-LDH-ura3HA as a template with primers of SEQ ID NO: 40 and SEQ ID NO: 41 to prepare a nde1 gene deletion cassette.

2.2 Preparation of Saccharomyces cerevisiae Strain from which Nde1 is Deleted

A mutant strain of the Δtrp1::Idh strain (KCTC12415BPΔtrp1::Idh), in which nde1 is deleted, was prepared in the same manner as follows.

The Δtrp1::Idh strain (KCTC12415BPΔtrp1:Idh) was spread onto a YPD agar plate (including 10 g of yeast extract, 20 g of peptone, and 20 g of glucose) and incubated for 24 hours at 30° C., and then, a colony obtained therefrom was inoculated in about 10 ml of a YPD liquid medium and cultured for 18 hours at 30° C. The sufficiently grown culture solution was inoculated in about 50 ml of a YPD liquid medium contained in a 250 ml-flask at a concentration of 1% (v/v) and in an incubator vortexing at a rate of about 230 rpm and at 30° C. After about 4 to 5 hours, when the OD₆₀₀ reached about 0.5, the culture was centrifuged at a rate of about 4,500 rpm for about 10 minutes to harvest cells, and the cells were resuspended in a lithium acetate solution at a concentration of about 100 mM. Then, the suspended cells were centrifuged at a rate of about 4,500 rpm for about 10 minutes to harvest cells, and the harvested cells were re-suspended in a lithium acetate solution at a concentration of about 1 M including about 15% of glycerol.

In order to delete an nde1 gene, the nde 1 deletion cassette was mixed with 50% of polyethylene glycol and single stranded carrier DNA, 100 ul of the re-suspension solution containing the water-soluble competent cells was added thereto, and reacted in a water tub for about 1 hour at 42° C. Then, the culture solution was spread on a uracil-free minimal agar plate (including YSD, 6.7 g/L of yeast nitrogen base without amino acids, and 1.4 g/L of amino acid dropout mix (-ura)) and grown for about 24 hours or more at 30° C. Ten colonies grown on the plate were selected, spread onto the fresh uracil-free minimal agar plate, and at the same time, inoculated into a liquid medium including the same components contained in the uracil-free minimal agar plate to isolate the genomic DNA from the above mutant strains by using a commonly used kit (Gentra Puregene Cell kit, Qiagen, USA). In order to confirm deletion of the nde 1 gene, a PCR was performed using the isolated genomic DNA of the mutant strain as a template with primers of SEQ ID NOs: 42 and 43, and then, electrophoresis was performed on the obtained PCR product to confirm nde1 deletion. As a result, the obtained strain was KCTC12415BPΔtrp1::IdhΔnde1+ura3.

Also, for additional gene deletion using the gene deletion vector, a selection marker URA 3 gene, which was used for the deletion of the nde1 gene, was removed by using a URA3 pop-out method. That is, KCTC12415BPΔtrp1:IdhΔnde1+ura3 was inoculated in about 10 ml of a YPD liquid medium, cultured for about 18 hours at 30° C., spread on a 5-FOA agar plate (including YSD, 6.7 g/L of yeast nitrogen base without amino acids, 1.4 g/L of amino acid dropout mix, and 1 μg/L of 5-fluoroorotic acid), and cultured for about 24 hours at 30° C. Ten colonies (URA3 pop-out strain) grown on the 5-FOA agar plate were selected, spread onto the fresh 5-FOA agar plate, and at the same time, cultured in a YPD liquid medium to isolate genomic DNA from the selected strain by using a commonly used kit (Gentra Puregene Cell kit, Qiagen, USA). In order to confirm deletion of the URA 3 gene by using the genomic DNA of the isolated URA3 pop-out strain as a template, a PCR was performed using primers of SEQ ID NOs: 42 and 43, and then electrophoresis was performed on the obtained PCR product to conform URA3 gene deletion. As a result, Δnde1 strain (KCTC12415BP Δtrp1::IdhΔnde1) was obtained.

2.3. Preparation of nde2 Gene Deletion Cassette

A vector for disrupting a nde 1 gene, i.e., pUC57-CCW12p-LDH-ura3HA prepared in Example 2.1 was used to delete a nde2 gene by using a homologous recombination method. In order to prepare a nde2 gene deletion cassette, PCR was performed using the prepared pUC57-CCW12p-LDH-ura3HA as a template with primers of SEQ ID NOs: 44 and 45.

2.4. Preparation of S. cerevisiae Strain from which nde1 and nde2 are Deleted

An nde2 deleted strain of Δnde1 (KCTC12415BPΔtrp1::IdhΔnde1) was prepared in the same manner as follows.

The Δnde1 strain was treated with a lithium acetate solution in the same manner as in Example 2.2, thereby obtaining competent cells.

In order to delete an nde2 gene, the nde2 deletion cassette prepared in Example 2.3 was mixed with 50% of polyethylene glycol and a single stranded carrier DNA, and 100 ul of the re-suspension solution for the competent cells was added thereto to obtain colonies on a uracil-free minimal agar plate in the same manner as in Example 2.2. Ten colonies (i.e., mutant strains) grown on the uracil-free minimal agar plate were selected, spread onto a fresh uracil-free minimal agar plate, and at the same time, inoculated into a liquid medium including the same components as those contained in the uracil-free minimal agar plate to isolate the genomic DNA from the mutant strains above by using a commonly used kit (Gentra Puregene Cell kit, Qiagen, USA). In order to confirm nde 2 gene deletion, a PCR was performed using the isolated genomic DNA of the mutant strain as a template with primers of SEQ ID NOs: 46 and 47, and then, electrophoresis was performed on the obtained PCR product to confirm nde2 gene deletion. As a result, the obtained strain was referred to as KCTC12415BPΔtrp1::IdhΔnde1Δnde2+ura3.

Also, for additional gene deletion, a selection marker URA3 gene, which was used for nde2 gene deletion, was removed from Saccharomyces cerevisiae KCTC12415BP (Δtrp1::IdhΔnde1Δnde2+ura3) by using a URA3 pop-out method in the same manner as in Example 2.2. In order to confirm URA 3 gene deletion by using the genomic DNA of the isolated URA3 pop-out strain as a template, a PCR was performed using primers of SEQ ID NOs: 46 and 47, and then, electrophoresis was performed on the obtained PCR product to confirm URA3 gene deletion. As a result, the obtained strain was Δnde1Δnde2 (KCTC12415BPΔtrp1::IdhΔnde1Δnde2).

### Example 3

### Preparation of Saccharomyces cerevisiae from which Mpc1 is Deleted

3.1. Preparation of mpc1 Gene Deletion Cassette

In order to delete a mpc1 gene by using a homologous recombination method, PCR was performed using the pUC57-ura3HA of Example 2 as a template with primers of SEQ ID NOs: 48 and 49 (i.e., mpc1_del_F and mpc1_del_R) to prepare a mpc1 gene deletion cassette.

3.2. Preparation of Saccharomyces cerevisiae from which Mpc1 is Deleted

A mpc1 deleted strain of the Δnde1Δnde2 (KCTC12415BPΔtrp1:Idh Δnde1Δnde2) was prepared in the same manner as follows.

The Δnde1Δnde2 strain of Example 2 was treated with a lithium acetate solution in the same manner as in Example 2.2, thereby obtaining re-suspension solution of competent cells.

In order to delete a mpc1 gene, the mpc 1 gene deletion cassette prepared in Example 3.1 was mixed with 50% of polyethylene glycol and single stranded carrier DNA, 100 ul of the re-suspension solution of competent cells was added thereto to obtain colonies on a uracil-free minimal agar plate in the same manner as in Example 2.2. Ten colonies (i.e., mutant strains) grown on the uracil-free minimal agar plate were selected, spread onto a fresh uracil-free minimal agar plate, and at the same time, inoculated into a liquid medium including the same components as those contained in the uracil-free minimal agar plate to isolate the genomic DNA from the mutant strains above by using a commonly used kit (Gentra Puregene Cell kit, Qiagen, USA). In order to confirm mpc1 gene deletion, a PCR was performed using the isolated genomic DNA of the mutant strain as a template with primers of SEQ ID NOs: 52 and 53 (i.e., mpc1_up_F and mpc1_down_R), and then, electrophoresis was performed on the obtained PCR product to confirm mpc1 gene deletion. As a result, the obtained strain was KCTC12415BPΔtrp1::Idh Δnde1Δnde2Δmpc1+ura3.

Also, for additional gene deletion, a selection marker URA3 gene, which was used for the mpc1 gene deletion, was removed from Saccharomyces cerevisiae KCTC12415BP (Δtrp1::IdhΔnde1Δnde2 Δmpc1+ura3) by using a URA3 pop-out method in the same manner as in Example 2.2. In order to confirm URA3 deletion by using the genomic DNA of the isolated URA3 pop-out strain as a template, a PCR was performed using primers of SEQ ID NOs: 52 and 53, and then, electrophoresis was performed on the obtained PCR product to confirm URA3 gene deletion. As a result, the obtained strain was Δmpc1 (KCTC12415BPΔtrp1::IdhΔnde1Δnde2Δmpc1).

### Example 4

### Preparation of Saccharomyces cerevisiae Strain from which mpc2 is Deleted

4.1. Preparation of mpc2 Gene Deletion Cassette

In order to delete an mpc2 gene by using a homologous recombination method, a PCR was performed using the pUC57-ura3HA of Example 2 as a template with primers of SEQ ID NOs: 50 and 51 (i.e., mpc2_del_F and mpc2_del_R) to prepare an mpc2 gene deletion cassette.

4.2. Preparation of Saccharomyces cerevisiae Strain from which mpc2 is Deleted

An mpc2 deleted strain of the Δnde1Δnde2 (KCTC12415BPΔtrp1::IdhΔnde1Δnde2) was prepared in the same manner as follows.

The Δnde1Δnde2 strain was treated with a lithium acetate solution in the same manner as in Example 2.2, thereby obtaining a re-suspension solution of competent cells.

In order to delete an mpc2 gene, the mpc2 gene deletion cassette prepared in Example 4.1 was mixed with 50% of polyethylene glycol and single stranded carrier DNA, 100 ul of the re-suspension solution of competent cells was added thereto to obtain colonies on a uracil-free minimal agar plate in the same manner as in Example 2.2. Ten colonies (i.e., mutant strains) grown on the uracil-free minimal agar plate were selected, spread onto a fresh uracil-free minimal agar plate, and at the same time, inoculated into a liquid medium including the same components as those contained in the uracil-free minimal agar plate to isolate the genomic DNA from the mutant strains above by using a commonly used kit (Gentra Puregene Cell kit, Qiagen, USA). In order to confirm mpc2 gene deletion, a PCR was performed using the isolated genomic DNA of the mutant strain as a template with primers of SEQ ID NOs: 54 and 55 (i.e., mpc2_up_F and mpc2_down_R), and then, electrophoresis was performed on the obtained PCR product to confirm mpc2 gene deletion. As a result, the obtained strain was KCTC12415BPΔtrp1::Idh Δnde1Δnde2Δmpc2+ura3.

Also, for additional gene deletion, a selection marker URA3 gene, which was used for the mpc2 gene deletion, was removed from Saccharomyces cerevisiae KCTC12415BP (Δtrp1::IdhΔnde1Δnde2Δmpc2+ura3) by using a URA3 pop-out method in the same manner as in Example 2.2. In order to confirm URA3 deletion by using the genomic DNA of the isolated URA3 pop-out strain as a template, a PCR was performed using primers of SEQ ID NOs:54 and 55, and then, electrophoresis was performed on the obtained PCR product to confirm URA3 gene deletion. As a result, the obtained strain was referred to as Δmpc2 (KCTC12415BPΔtrp1::Idh Δnde1Δnde2Δmpc2).

### Example 5

### Preparation of Saccharomyces cerevisiae Strain from which mpc1 and mpc2 are Deleted

In order to prepare a mutant of Δmpc1Δmpc2 from which a mpc1 gene and a mpc2 gene are deleted as follows, the Δmpc1 strain (KCTC12415BPΔtrp1:IdhΔnde1Δnde2Δmpc1) of Example 3.2 was treated with a lithium acetate solution in the same manner as in Example 2.2, thereby obtaining competent cells.

In order to delete an mpc2 gene, the mpc2 deletion cassette prepared in Example 4.1 was mixed with 50% of polyethylene glycol and single stranded carrier DNA, and 100 ul of the re-suspension solution of competent cells was added thereto to obtain colonies on a uracil-free minimal agar plate in the same manner as in Example 2.2. Ten colonies (i.e., mutant strains) grown on the uracil-free minimal agar plate were selected, spread onto a fresh uracil-free minimal agar plate, and at the same time, inoculated into a liquid medium including the same components as those contained in the uracil-free minimal agar plate to isolate the genomic DNA from the mutant strains above by using a commonly used kit (Gentra Puregene Cell kit, Qiagen, USA). In order to confirm mpc2 gene deletion, a PCR was performed using the isolated genomic DNA of the mutant strain as a template with primers of SEQ ID NOs: 54 and 55 (i.e., mpc2_up_F and mpc2_down_R), and then, electrophoresis was performed on the obtained PCR product to confirm mpc2 gene deletion. As a result, the obtained strain was referred to as KCTC12415BPΔtrp1::Idh Δnde1Δnde2Δmpc1Δmpc2+ura3.

Also, a selective marker URA 3 gene was removed from KCTC12415BPΔtrp1::IdhΔnde1Δnde2Δmpc1Δmpc2+ura3 by using a URA3 pop-out in the same manner as in Example 2.2. In order to confirm URA 3 gene deletion by using the genomic DNA of the isolated URA3 pop-out strain as a template, a PCR was performed using primers of SEQ ID NOs: 54 and 55, and then, electrophoresis was performed on the obtained PCR product to confirm URA3 gene deletion. As a result, the obtained strain was Δmpc1Δmpc2 (KCTC12415BPΔtrp1::Idh Δnde1Δnde2Δmpc1Δmpc2).

### Example 6

### Production of Ethanol and Lactate Using Each of a Strain in which a MPC Gene is Disrupted

Each of the strains Δnde1Δnde2, Δmpc1, Δmpc2, and Δmpc1Δmpc2 that were prepared in Examples 2 to 5, respectively, was spread onto a YPD agar plate and cultured at 30° C. for 24 hours or more, and then a colony obtained therefrom was inoculated in 50 ml of a YPD liquid medium including 40 g/L of glucose and cultured at 30° C. for 16 hours under aerobic conditions. Here, the fermentation was performed as follows. An amount of the culture that has a cell concentration of 5.0 at a light absorbance (optical density) of 600 nm (OD 5.0) as measured by using a spectrophotometer in the 50 ml of the culture medium was quantified and centrifuged, and the supernatant was removed. Then, the cells were re-suspended and re-inoculated in 50 ml of a new YPD liquid medium including 80 g/L of glucose, and then incubated.

The cells were cultured in an incubator at a temperature of 30° C. for 24 hours or more, while stirring at a rate of about 90 rpm. During the incubation, samples were periodically obtained from the flask, and the obtained samples were centrifuged at a rate of about 13,000 rpm for about 10 minutes. Then, concentrations of ethanol of the supernatant, lactate, and glucose were analyzed by using high-performance liquid chromatography (HPLC).

As shown in Table 1, L-lactate production of the Δmpc1 strain was increased from 31.0 g/L to 35.1 g/L compared to the Δnde1Δnde2 strain used as a reference strain, and ethanol production of the Δmpc1 strain was increased from 19.7 g/L to 23.4 g/L compared to the Δnde1Δnde2 strain used as a reference strain. In addition, L-lactate production of the Δmpc1Δmpc2 strain was increased from 31.0 g/L to 35.6 g/L compared to the Δnde1Δnde2 strain used as a reference strain, and ethanol production of the Δmpc1Δmpc2 strain was increased from 19.7 g/L to 23.2 g/L compared to the Δnde1Δnde2 strain used as a reference strain. As a result, it was confirmed that the strains from which the MPC gene was deleted had increased productivities and increased yields of lactate and ethanol.

TABLE 1

| | | | | | | | LA + |
|---|---|---|---|---|---|---|---|
| | | | LA | Yield | EtOH | Yield | EtOH |
| Strain | Characteristic | OD | (g/L) | (%) | (g/L) | (%) | (g/L) |
| Δnde1Δ | KCTC12415BPΔ | 18.1 | 31.0 | 46.8 | 19.7 | 42.9 | 50.7 |
| nde2 | trp1::ldhΔnde1Δ | | | | | | |
| | nde2 | | | | | | |
| Δmpc1 | KCTC12415BPΔt | 16.2 | 35.1 | 46.2 | 23.4 | 45.3 | 58.5 |
| | rp1::ldhΔnde1Δn | | | | | | |
| | de2Δmpc1 | | | | | | |
| Δmpc2 | KCTC12415BPΔt | 18.9 | 33.1 | 43.8 | 24.2 | 49.4 | 57.3 |
| | rp1::ldhΔnde1Δn | | | | | | |
| | de2Δmpc2 | | | | | | |
| Δmpc1Δ | KCTC12415BPΔt | 21.6 | 35.6 | 46.7 | 23.2 | 43.2 | 58.8 |
| mpc2 | rp1::ldhΔnde1Δn | | | | | | |
| | de2Δmpc1Δmpc2 | | | | | | |

In regard to Table 1 above, the strains were cultured in a 50 ml flask for about 30 hours. Here, LA denotes lactate and EtOH denotes ethanol.

### Example 7

### Production of Ethanol and Lactate Using ΔMpc1 ΔMpc2 Strain

The Δmpc1Δmpc2 strain prepared in Example 5 was spread on a YPD agar plate and cultured at 30° C. for 24 hours or more, and then a colony obtained therefrom was inoculated in 100 ml of a YPD liquid medium including 80 g/L of glucose and cultured at 30° C. for 16 hours under aerobic conditions. Here, 100 ml of the strain culture solution was separately inoculated in a bioreactor containing 1 L of a synthetic medium and cultured.

The culture was performed with initial concentrations of 60 g/L of glucose and 20 g/L of an yeast extract at 30° C. During the culture, pH was maintained at about pH 5 for up to 16 hours, at about pH 4.5 for up to 24 hours, and at about pH 3.0 for up to 60 hours by using 5N Ca(OH)₂, and a concentration of the glucose was maintained at 20 g/L. Additional synthetic medium compositions include 50 g/L of K₂HPO₄, 10 g/L of MgSO₄, 0.1 g/L of tryptophan, and 0.1 g/L of histidine in addition to the glucose.

A cell concentration in the culture solution was measured by using a spectrophotometer. During the culture, samples were periodically obtained from a bioreactor, and the obtained samples were centrifuged at a rate of 13,000 rpm for 10 minutes. Then, concentrations of metabolites of the supernatant, lactate, and glucose were analyzed by using HPLC.

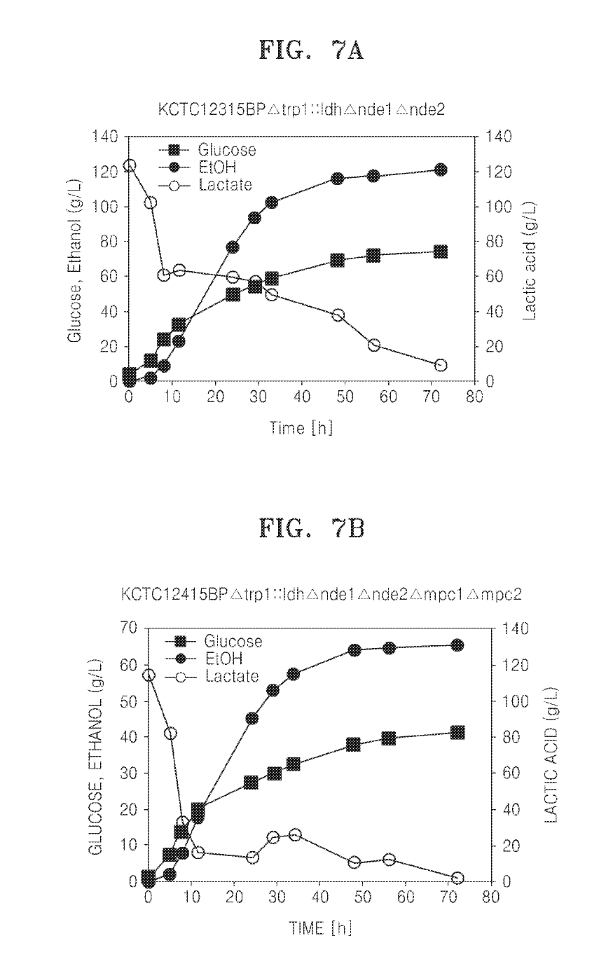

As shown in FIG. 7, the Δmpc1Δmpc2 strain (FIG. 7B) exhibited excellent lactate productivity, and a lactate yield thereof also was increased compared to the Δnde1Δnde2 strain (FIG. 7A) used as a reference strain. The lactate production of the Δmpc1Δmpc2 strain was increased from 115.8 g/L to 128.2 g/L and the yield thereof was increased from 57.7% to 63.8% compared to the Δnde1Δnde2 strain used as a reference strain. Also, the Δmpc1 Δmpc2 strain exhibited increased ethanol productivity, and an ethanol yield thereof also was increased compared to the Δnde1Δnde2 strain as a reference strain. The ethanol production of the Δmpc1Δmpc2 strain was increased from 34.5 g/L to 37.8 g/L and the yield thereof was increased from 16.4% to 18.2% compared to the Δnde1Δnde2 strain used as a reference strain.

[Accession Number]

Research Center Name: Korean Collection for Type Cultures (KCTC)

Accession Number: KCTC12415BP

Accession Date: 2013 May 30

As described above, according to the one or more of the above embodiments of the present invention, a yeast cell may increase a level of pyruvate within a cytosol by reducing activity of a MPC, and accordingly a yeast cell may produce a pyruvate-based metabolite, which is converted from pyruvate, at a high concentration and a high yield. Also, according to the one or more of the above embodiments of the present invention, a method of producing a pyruvate-based metabolite may produce a pyruvate-based metabolite at a high concentration and a high yield.

It should be understood that the exemplary embodiments described herein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A genetically engineered yeast cell that produces a pyruvate-based metabolite from pyruvate,
wherein the genetically engineered yeast cell has a deletion or disruption mutation of a gene encoding MPC and activity of a mitochondrial pyruvate carrier (MPC) is reduced compared to a parent yeast cell not having a deletion or disruption mutation of the gene encoding MPC.

2. The yeast cell of claim 1, wherein the yeast cell belongs to the genus Saccharomyces, the genus Candida, the genus Shizosaccharomyces, the genus Kluyveromyces, the genus Pichia, the genus Issachenkia, or the genus Hansenula.

3. The yeast cell of claim 2, wherein the yeast cell belongs to the genus Saccharomyces.

4. The yeast cell of claim 1, wherein the MPC is MPC1, MPC2, MPC3, or a combination thereof.

5. The yeast cell of claim 1, wherein the MPC comprises a polypeptide having an amino acid sequence with a sequence identity of about 95% or greater with respect to an amino acid sequence selected from SEQ ID NO: 1, SEQ ID NO: 2, and SEQ ID NO: 3.

6. The yeast cell of claim 1, wherein the gene encoding the MPC has at least one nucleic acid sequence selected from SEQ ID NO: 4, SEQ ID NO: 5, and SEQ ID NO: 6.

7. The yeast cell of claim 1, wherein the pyruvate-based metabolite is at least one selected from lactate, ethanol, glycerol, acetate, formate, alanine, carbon dioxide, and hydrogen.

8. The yeast cell of claim 1, wherein the pyruvate-based metabolite is lactate.

9. The yeast cell of claim 8, wherein the genetically engineered yeast cell comprises a polypeptide that converts pyruvate into lactate, and the activity of the polypeptide is increased as compared to a parent yeast cell.

10. The yeast cell of claim 8, wherein the yeast cell comprises a polynucleotide encoding lactate dehydrogenase.

11. The yeast cell of claim 9, wherein the polypeptide converting pyruvate into lactate has an amino acid sequence with a sequence identity of about 95% or greater with respect to SEQ ID NO: 7.

12. The yeast cell of claim 9, wherein a polynucleotide encoding the polypeptide converting pyruvate into lactate has a nucleotide sequence of SEQ ID NO: 11.

13. The yeast cell of claim 8, wherein the genetically engineered yeast cell exhibits reduced activity of converting pyruvate into acetaldehyde, converting lactate into pyruvate, and/or converting dihydroxyacetone phosphate (DHAP) into glycerol-3-phosphate as compared to a parent yeast cell.

14. The yeast cell of claim 8, wherein the yeast cell contains a deletion or disruption mutation of a gene encoding a polypeptide for converting pyruvate into acetaldehyde, a gene encoding a polypeptide for converting lactate into pyruvate, a gene encoding a polypeptide for converting DHAP into glycerol-3-phosphate, or a combination thereof.

15. The yeast cell of claim 1, wherein the pyruvate-based metabolite is ethanol.

16. The yeast cell of claim 15, wherein the genetically engineered yeast cell exhibits increased activity of converting pyruvate into ethanol as compared to a parent yeast cell.

17. The yeast cell of claim 15, wherein the yeast cell comprises pyruvate decarboxylase (PDC), alcohol dehydrogenase (ADH), or a combination thereof.

18. The yeast cell of claim 15, wherein the genetically engineered yeast cell exhibits reduced activity of converting pyruvate into lactate, converting DHAP into glycerol-3-phosphate, or any combination thereof as compared to a parent yeast cell.

19. The yeast cell of claim 1, wherein the genetically engineered yeast cell exhibits reduced mitochondrial NADH dehydrogenase activity as compared to a parent yeast cell.

20. The yeast cell of claim 19, wherein the mitochondrial NADH dehydrogenase is NDE1, NDE2, NDE3, or a combination thereof.

21. The yeast cell of claim 19, wherein the mitochondrial NADH dehydrogenase has an amino acid sequence with a sequence identity of about 95% or greater with respect to SEQ ID NO: 26 or SEQ ID NO: 27.

22. A method of producing a pyruvate-based metabolite, the method comprising:
culturing the yeast cell of claim 1; and
collecting the pyruvate-based metabolite from the culture.

23. The method of claim 22, wherein the pyruvate-based metabolite is at least one selected from lactate, ethanol, glycerol, acetate, formate, alanine, carbon dioxide, and hydrogen.

24. The method of claim 22, wherein the culturing is performed under anaerobic conditions.

25. A method of preparing a yeast cell that produces a pyruvate metabolite from pyruvate, the method comprising disrupting or deleting one or more genes encoding a mitochondrial pyruvate carrier (MPC) protein in a yeast cell.

26. The method of claim 25, wherein the method comprises disrupting or deleting one or more genes encoding MPC1, MPC2, MPC3, or combination thereof.

27. A method of increasing the production efficiency of a pyruvate metabolite from pyruvate, the method comprising disrupting or deleting one or more genes encoding a mitochondrial pyruvate carrier (MPC) protein in a pyruvate metabolite-producing yeast cell, whereby the production efficiency of the pyruvate metabolite is increased.

28. The method of claim 25, wherein the method comprises disrupting or deleting one or more genes encoding MPC1, MPC2, MPC3, or combination thereof.
